SEQUENCE LISTING (10 sequences)

SEQ ID NO: 1 - protein, 5 aa, synthetic construct
GYNMN

SEQ ID NO: 2 - protein, 17 aa, synthetic construct
NIDPYYGGTSYNQKFKG

SEQ ID NO: 3 - protein, 9 aa, synthetic construct
MYHGNAFDY

SEQ ID NO: 4 - protein, 17 aa, synthetic construct
KSSQSLLNSGNLKNYLT

SEQ ID NO: 5 - protein, 7 aa, synthetic construct
WASTRKS

SEQ ID NO: 6 - protein, 9 aa, synthetic construct
QNDYSYPLT

SEQ ID NO: 7 - protein, 118 aa, synthetic construct
QVQLVQSGAEVKKPGASVKVSCKASGYTFTGYNMNWVRQAPGQGLEWMGNIDPYYGGTSYNQKFKGRVTMTIDKSTSTVYMELSSLRSEDTAVYYCARMYHGNAFDYWGQGTTVTVSS

SEQ ID NO: 8 - protein, 113 aa, synthetic construct
DIVMTQSPDSLAVSLGERATINCKSSQSLLNSGNLKNYLTWYQQKPGQPPKLLIYWASTRKSGVPDRFSGSGSGTDFTLTISSLQAEDVAVYYCQNDYSYPLTFGGGTKVEIK

SEQ ID NO: 9 - protein, 401 aa, synthetic construct
QVQLVQSGAEVKKPGASVKVSCKASGYTFTGYNMNWVRQAPGQGLEWMGNIDPYYGGTSYNQKFKGRVTMTIDKSTSTVYMELSSLRSEDTAVYYCARMYHGNAFDYWGQGTTVTVSSSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 220 aa, synthetic construct
DIVMTQSPDSLAVSLGERATINCKSSQSLLNSGNLKNYLTWYQQKPGQPPKLLIYWASTRKSGVPDRFSGSGSGTDFTLTISSLQAEDVAVYYCQNDYSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0258122 A1
(43) Pub. Date: Sep. 3, 2026

(54) STABLE PHARMACEUTICAL FORMULATION COMPRISING AN ANTI-CLDN18.2 ANTIBODY

(71) Applicants: SUZHOU TRANSCENTA THERAPEUTICS CO., LTD., Suzhou, Jiangsu (CN); TRANSCENTA HOLDING LIMITED, Grand Cayman (KY); TRANSCENTA THERAPEUTICS, INC., Princeton, NJ (US)

(72) Inventors: Fan ZHANG, Suzhou, Jiangsu (CN); Yudi HU, Suzhou, Jiangsu (CN)

(21) Appl. No.: 18/879,798

(22) PCT Filed: Jun. 29, 2023
(86) PCT No.: PCT/CN2023/103958

§ 371 Date: Dec. 29, 2024

(30) Foreign Application Priority Data
Jun. 30, 2022 (CN) 202210769128.9

(51) Int. Cl. C07K 16/28; A61K 47/10; A61K 47/12; A61K 47/18; A61K 47/22; A61K 47/26

(52) U.S. Cl. CPC C07K 16/28; A61K 47/10; A61K 47/12; A61K 47/183; A61K 47/22; A61K 47/26

(57) ABSTRACT

Provided is a pharmaceutical formulation. The pharmaceutical formulation comprises an anti-Claudin (CLDN) 18.2 antibody, a buffer, a stabilizer and a surfactant. The pharmaceutical formulation provided herein could maintain the stability of the anti-CLDN18.2 antibody after long-term storage, storage at high temperature (e.g., 40° C.) and/or multiple freezing and thawing cycles. Further provided is the use of the pharmaceutical formulation in the manufacture of a medicament for preventing and/or treating CLDN18.2 related diseases, especially cancers (e.g., CLDN 18.2 related cancers), and a method for preparing the pharmaceutical formulation.

### FIELD OF THE INVENTION

The present disclosure relates to a pharmaceutical formulation, and in particular, to a stable pharmaceutical formulation comprising an anti-CLDN18.2 antibody. The present disclosure also relates to a method of preparing the pharmaceutical formulation and uses thereof.

### BACKGROUND

Studies have shown that CLDN18.2 belongs to members of the Claudin (CLDN) family and is one of subtypes of CLDN18. The CLDN18.2 is a highly selective gastric lineage marker and is highly expressed in gastric cancer, esophagus cancer, pancreatic cancer, lung cancer and a variety of other cancer types. The CLDN18.2 protein structurally includes 4 transmembrane regions and 2 extracellular rings, and is a transmembrane protein located on the surface of a cell membrane. As a protein on the surface of the cell membrane, the CLDN18.2 is allowed to bind to an antibody due to the exposed extracellular structure, thus becoming an ideal target for the development of therapeutic antibodies. Anti-CLDN18.2 antibodies can specifically recognize and bind to CLDN18.2 molecules on the surfaces of tumor cells, leading to antibody-dependent cytotoxicity (ADCC) and complement dependent cytotoxicity (CDC), thus inducing apoptosis and inhibition of cell proliferation, removing cancer cells, and controlling diseases.

Antibody molecules have complex multi-level protein structures and are prone to physical binding, which may cause undesired immune responses, or may become unsafe for patients due to clogging the syringe or pump during administration. Thus, one of the long-standing problems with liquid formulations of antibodies is stability issues due to aggregation.

Therefore, there is need for pharmaceutical formulations of an anti-CLDN18.2 antibody having stability and consistent quality.

### SUMMARY

The present disclosure provides a stable pharmaceutical formulation comprising an anti-CLDN18.2 antibody that retains uniformity and stability after long-term storage, treatment (e.g., storage) at high temperature (e.g., 40° C.) and/or multiple freezing and thawing cycles.

In one aspect, the present disclosure provides a pharmaceutical formulation, wherein the pharmaceutical formulation comprises an anti-CLDN18.2 antibody and a buffer, where the buffer is an acetic acid buffer or a histidine buffer, and the pH value is 4.5-6.0. In some embodiments, the concentration of the buffer in the pharmaceutical formulation is 5 mM-50 mM, or 10 mM-30 mM.

In some embodiments, the pharmaceutical formulation further comprises a stabilizer.

In some embodiments, the concentration of the stabilizer in the pharmaceutical formulation is 1% (w/v)-20% (w/v), or 1% (w/v)-10% (w/v).

In some embodiments, the stabilizer is selected from the group consisting of: sucrose, trehalose, and sorbitol.

In some embodiments, the stabilizer is sucrose or trehalose, and the concentration of the sucrose or the trehalose in the pharmaceutical formulation is 5% (w/v)-10% (w/v). In other embodiments, the stabilizer is sorbitol, and the concentration of the sorbitol in the pharmaceutical formulation is 2% (w/v)-8% (w/v).

In some embodiments, the pharmaceutical formulation further comprises a surfactant.

In some embodiments, the concentration of the surfactant in the pharmaceutical formulation is 0.005% (w/v)-0.4% (w/v), or 0.01% (w/v)-0.2% (w/v).

In some embodiments, the surfactant is selected from the group consisting of: polysorbate 80 and poloxamer 188.

In some embodiments, the surfactant is polysorbate 80, and the concentration of the polysorbate 80 in the pharmaceutical formulation is 0.01% (w/v)-0.1% (w/v). In other embodiments, the surfactant is poloxamer 188, and the concentration of the poloxamer 188 in the pharmaceutical formulation is 0.05% (w/v)-0.2% (w/v).

In some embodiments, the pharmaceutical formulation further comprises a chelating agent.

In some embodiments, the concentration of the chelating agent in the pharmaceutical formulation is 30 μM-350 μM, or 40 μM-60 μM.

In some embodiments, the chelating agent is selected from the group consisting of: EDTA, DTPA, IDHA, EDDHA, and HBED.

In some embodiments, the concentration of the anti-CLDN18.2 antibody in the pharmaceutical formulation is 1 mg/ml-200 mg/ml.

In some embodiments, the concentration of the anti-CLDN18.2 antibody in the pharmaceutical formulation is 20 mg/ml-40 mg/ml.

In some embodiments, the anti-CLDN18.2 antibody comprises a heavy chain CDR1 (HCDR1) as set forth in SEQ ID NO: 1, an HCDR2 as set forth in SEQ ID NO: 2 and an HCDR3 as set forth in SEQ ID NO: 3, and a light chain CDR1 (LCDR1) as set forth in SEQ ID NO: 4, an LCDR2 as set forth in SEQ ID NO: 5 and an LCDR3 as set forth in SEQ ID NO: 6.

In some embodiments, the anti-CLDN18.2 antibody comprises a CDR1, a CDR2 and a CDR3 of the heavy chain variable region as set forth in SEQ ID NO: 7, and a CDR1, a CDR2 and a CDR3 of the light chain variable region as set forth in SEQ ID NO: 8.

In some embodiments, the anti-CLDN18.2 antibody comprises a heavy chain variable region as set forth in SEQ ID NO: 7 and a light chain variable region as set forth in SEQ ID NO: 8.

In some embodiments, the anti-CLDN18.2 antibody comprises a heavy chain as set forth in SEQ ID NO: 9 and a light chain as set forth in SEQ ID NO: 10.

In some embodiments, the pharmaceutical formulation comprises an anti-CLDN18.2 antibody, a buffer, a stabilizer, and a surfactant, wherein the buffer is an acetic acid buffer, the stabilizer is sucrose or trehalose, the surfactant is polysorbate 80, and the pH value is about 4.5-6.0.

In some embodiments, the concentration of the anti-CLDN18.2 antibody in the pharmaceutical formulation is 20 mg/ml-40 mg/ml, the concentration of the acetic acid buffer in the pharmaceutical formulation is 10 mM-30 mM, the concentration of the sucrose or the trehalose in the pharmaceutical formulation is 5% (w/v)-10% (w/v), and/or the concentration of the polysorbate 80 in the pharmaceutical formulation is 0.01% (w/v)-0.2% (w/v).

In some embodiments, the concentration of the anti-CLDN18.2 antibody in the pharmaceutical formulation is about 30 mg/ml, the concentration of the acetic acid buffer in the pharmaceutical formulation is about 20 mM, the concentration of the sucrose or the trehalose in the pharmaceutical formulation is about 9% (w/v), the concentration of the polysorbate 80 in the pharmaceutical formulation is 0.01% (w/v)-0.1% (w/v), and the pH value is about 5.0-5.5.

In some embodiments, the stabilizer is sucrose, the concentration of the polysorbate 80 in the pharmaceutical formulation is about 0.05% (w/v), and the pH value is about 5.3.

In some embodiments, the pharmaceutical formulation further comprises EDTA, and the concentration of the EDTA in the pharmaceutical formulation is 40 μM-60 μM.

In some embodiments, the concentration of the EDTA in the pharmaceutical formulation is about 50 μM.

In some embodiments, the pharmaceutical formulation comprises an anti-CLDN18.2 antibody, a buffer, a stabilizer and a surfactant, wherein the buffer is a histidine buffer, the stabilizer is sucrose or sorbitol, and the surfactant is polysorbate 80 or poloxamer 188.

In some embodiments, the stabilizer is sucrose, and the concentration of the sucrose in the pharmaceutical formulation is 6% (w/v)-12% (w/v). In other embodiments, the stabilizer is sorbitol, and the concentration of the sorbitol in the pharmaceutical formulation is 2% (w/v)-8% (w/v).

In some embodiments, the surfactant is polysorbate 80, and the concentration of the polysorbate 80 in the pharmaceutical formulation is 0.01% (w/v)-0.1% (w/v). In other embodiments, the surfactant is poloxamer 188, and the concentration of the poloxamer 188 in the pharmaceutical formulation is 0.05% (w/v)-0.2% (w/v).

In another aspect, the present disclosure provides a method for preparing a pharmaceutical formulation, comprising: (1) providing a formulation solvent and an anti-CLDN18.2 antibody stock solution, where the formulation solvent comprises a buffer, a stabilizer and optionally a surfactant; and (2) subjecting the anti-CLDN18.2 antibody stock solution to solvent exchange with the formulation solvent to obtain the pharmaceutical formulation described herein.

In some embodiments, the anti-CLDN18.2 antibody stock solution used in the method described herein comprises a chelating agent (e.g., EDTA), the formulation solvent does not comprise a chelating agent, and the pharmaceutical formulation comprises substantially no chelating agent (e.g., after filtration). In other embodiments, the formulation solvent comprises a buffer and a stabilizer and does not comprise a surfactant.

In other embodiments, the formulation solvent used in the method described herein comprises a buffer, a stabilizer and a chelating agent (e.g., EDTA).

In other embodiments, the surfactant is added after the anti-CLDN18.2 antibody stock solution has been subjected to solvent exchange with the formulation solvent to obtain the pharmaceutical formulation described herein.

In some embodiments, the acetic acid buffer used in the method described herein is an acetic acid-sodium acetate buffer; and the histidine buffer is a histidine-histidine hydrochloride buffer.

In another aspect, the present disclosure provides use of the pharmaceutical formulation described herein in the manufacture of a medicament for preventing and/or treating CLDN18.2 related diseases.

In some embodiments, the CLDN18.2 related diseases are selected from the group consisting of: gastric cancer, adenocarcinoma of the gastroesophageal junction, lung cancer, bronchogenic carcinoma, bone cancer, hilarcholangiocarcinoma, pancreatic cancer, breast cancer, liver cancer, ovarian cancer, testicular cancer, kidney cancer, bladder cancer, head and neck cancer, spinal cancer, brain cancer, cervical cancer, uterine cancer, endometrial cancer, colon cancer, colorectal cancer, rectal cancer, anal cancer, esophageal carcinoma, gastrointestinal cancer, skin cancer, prostate cancer, pituitary carcinoma, vaginal cancer, thyroid cancer, glioblastoma, astrocytoma, melanoma, myelodysplastic syndrome, sarcoma, teratoma or adenocarcinoma.

### BRIEF DESCRIPTION OF THE DRAWINGS

FIG. 1 shows protein stability data measured by SEC in a surfactant screening experiment using poloxamer 188 as a surfactant at three different concentrations respectively (formulations F14, F15 and F16).

FIG. 2 shows anti-CLDN18.2 antibody protein stability data measured by CEX in a surfactant screening experiment using poloxamer 188 as a surfactant at three different concentrations respectively (formulations F14, F15 and F16).

FIG. 3 shows protein stability data measured by CE-SDS in a surfactant screening experiment using poloxamer 188 as a surfactant at three different concentrations respectively (formulations F14, F15 and F16).

FIG. 4 shows protein stability data measured by SEC in an excipient screening experiment using sorbitol and sucrose respectively (formulations F21 and F22, respectively) as a stabilizer.

FIG. 5 shows protein stability data measured by CEX in an excipient screening experiment using sorbitol and sucrose respectively (formulations F21 and F22, respectively) as a stabilizer;

FIG. 6 shows protein stability data measured by CE-SDS in an excipient screening experiment using sorbitol and sucrose respectively (formulations F21 and F22, respectively) as a stabilizer;

FIG. 7 shows a protection effect of disodium edetate (disodium EDTA) on preventing polysorbate 80 (PS80) from degradation; and

FIG. 8 shows a protection effect of disodium edetate (disodium EDTA) on the protein stability as measured by NR CE-SDS.

### DETAILED DESCRIPTION

The following descriptions of the present disclosure are merely intended to describe various embodiments of the present disclosure. The specific examples described shall not be construed as limitations of the scope of the present disclosure. Various equivalent substitutions, alterations or variations may be made by persons skilled in the art without departing from the spirit and essence of the present disclosure, and it shall be understood that all these equivalent embodiments are also included herein. All documents cited herein, including publications, patents and patent applications, are incorporated herein by reference. When the method referred to in the disclosure includes two or more qualified steps, the steps defined may be performed in any order or simultaneously (unless the possibility is precluded in the context). Moreover, the method may include one or more of other steps, which may be performed before any steps defined, between two steps defined or after all the steps defined (unless the possibility is precluded in the context).

The reference to “about” (a value or a range of values) herein includes examples of the value or the range of values. For example, “about X” includes “X”. In general, the term “about” refers to the value of a variable, all values that are within an experimental error range of the variable (e.g., within 95% confidence interval of a mean) or all values that are within 10% of the variable, based on greater values. For example, “about X” includes “110%×X”, “109%×X”, “108%×X”, “107%×X”, “106%×X”, “105%×X”, “104%×X”, “103%×X”, “102%×X”, “101%×X”, “99%×X”, “98%×X”, “97%×X”, “96%×X”, “95%×X”, “94%×X”, “93%×X”, “92%×X”, “91%×X” or “90%×X”.

The term “at least” and a number thereafter are used herein for indicating the beginning of a range that begins with the number (which may be a range with or without an upper limit, depending on the variable defined). For example, “at least 1” indicates 1 or a value greater than 1. The term “at most” and a number thereafter are used herein for indicating the end of a range that ends with the number (which may be a range with 1 or 0 as a lower limit or a range without a lower limit, depending on the variable defined). For example, “at most 4” indicates 4 or a value less than 4, and “at most 40%” indicates 40% or a value less than 40%. In the present disclosure, when a range is set as “(first number) to (second number)” or “(first number)-(second number)”, it indicates that the lower limit of the range is the first number, and the upper limit is the second number. For example, 5-50 mg/mL indicates a range with 5 mg/mL as a lower limit and 50 mg/mL as an upper limit. The term “less than” or “greater than” a value used herein includes the value.

### Antibody

As used herein, the term “antibody” includes any immunoglobulins, monoclonal antibodies, polyclonal antibodies, multivalent antibodies, bivalent antibodies, monovalent antibodies, multispecific antibodies or bispecific antibodies that bind to specific antigens. A complete antibody includes two heavy chains and two light chains. Each heavy chain consists of a heavy chain variable region (V_{H}), a heavy chain first constant region (C_{H1}), a heavy chain second constant region (C_{H2}) and a heavy chain third constant region (C_{H3}). Each light chain consists of a light chain variable region (V_{L}) and a light chain constant region (C_{L}). The V_{H} region of the heavy chain and the V_{L} region of the light chain each has three complementary determinant regions (CDRs), which are interposed between flanking stretches known as framework regions (FR). The framework regions are more highly conserved than the CDRs and form a scaffold to support the hypervariable loops. The 6 CDRs of one heavy chain and one light chain together constitute an antigen-binding site of an antibody to determine the specificity of the antibody. The antibody described herein also includes fragments or derivatives having an antigen-binding function of a complete antibody. The fragments or the derivatives have the same antigen-binding specificity as the complete antibody, but the binding affinity of the fragments or the derivatives to a specific antigen may be the same or different from that of the complete antibody.

In some embodiments, the antibody described herein includes an antigen-binding fragment. The antigen-binding fragment refers to one or more types of antibody fragments that retain the binding specificity to an antigen. Examples of the antigen-binding fragment include, but are not limited to, (i) an Fab fragment, which is a monovalent fragment consisting of V_{L}, V_{H}, C_{L} and C_{H1} domains; (ii) an Fab′ fragment, which is an Fab fragment that includes a portion of a hinge region; (iii) an F (ab′)₂ fragment, which is a bivalent fragment containing 2 Fab fragments connected by a disulfide bond in the hinge region; (iv) an Fd fragment consisting of V_{H} and C_{H1} domains; (v) an Fv fragment consisting of V_{L} and V_{H} domains of a single arm of an antibody; (vi) a dAb fragment (Ward et al., Nature 341: 544-546 (1989); PCT disclosure WO 90/05144), which comprises a single variable domain; (vii) an isolated CDR; and (viii) a single-stranded Fv fragment, which is a monovalent fragment formed by connection between V_{L} and V_{H} domains directly or via a peptide chain (Huston J S et al., Proc Natl Acad Sci USA, 85: 5879 (1988)).

In some embodiments, the antibody described herein includes a chimeric antibody with a portion of heavy chain and/or light chain being identical or homologous to corresponding sequences of an antibody that is derived from a particular species or belong to a particular antibody class or subclass, and the remaining portion of the chains being identical or homologous to corresponding sequences of an antibody that is derived from another class or belong to another antibody class or subclass and fragments thereof, provided that it has desired functional activity.

In some embodiments, the antibody described herein includes a humanized antibody. The humanized form of a non-human (e.g., mouse) antibody can be a chimeric immunoglobulin, an immunoglobulin chain or fragments thereof (e.g., Fv, Fab, Fab′, F (ab′) 2 or other antigen-binding sequences of an antibody) that comprises minimal sequences obtained from a non-human immunoglobulin. In some examples, a humanized antibody may be a CDR grafted antibody, wherein amino acid sequences of human CDRs are introduced into amino acid sequences of non-human V_{H} and V_{L} to replace amino acid sequences of corresponding non-human CDRs. In other examples, the majority of the amino acid sequences of a humanized antibody may be derived from a human immunoglobulin (i.e., a receptor antibody), wherein the amino acid residues of CDRs of the receptor antibody are replaced by the amino acid residues of CDRs of a non-human (e.g., mouse, rat and rabbit) antibody having desired specificity, affinity and capability. Usually, a humanized antibody comprises at least one, generally two, variable domains, wherein all or substantially all of the CDR sequences are from a non-human immunoglobulin, and all or substantially all of framework region (FR) sequences are from a human immunoglobulin. In some examples, residues in the framework regions of variable regions of a human immunoglobulin are replaced by corresponding non-human residues. Moreover, a humanized antibody may include residues that exist neither in the original antibody nor in the sequences of introduced CDRs or framework regions.

The anti-CLDN18.2 antibody described herein refers to an antibody that can specifically bind to the CLDN18.2 protein.

The CLDN18.2 protein described herein refers to a Claudin-18 splice variant 2 derived from mammals, such as primates (e.g., humans and monkeys) and rodents (e.g., mice). In some embodiments, the CLDN18.2 is a human CLDN18.2. An exemplary sequence of the human CLDN18.2 comprises a human CLDN18.2 protein (NCBI Ref Seq No. NP_001002026.1). The CLDN18.2 may be expressed in cancer cells. In one embodiment, the CLDN18.2 is expressed on the surfaces of cancer cells.

In some embodiments, the anti-CLDN18.2 antibody described herein comprises a heavy chain variable region (V_{H}). The heavy chain variable region (V_{H}) comprises a CDR1 as set forth in SEQ ID NO: 1, a CDR2 as set forth in SEQ ID NO: 2 and/or a CDR3 as set forth in SEQ ID NO: 3.

In some embodiments, the anti-CLDN18.2 antibody described herein comprises a light chain variable region (V_{L}). The light chain variable region (V_{L}) comprises a CDR1 as set forth in SEQ ID NO: 4, a CDR2 as set forth in SEQ ID NO: 5 and/or a CDR3 as set forth in SEQ ID NO: 6.

In some embodiments, the anti-CLDN18.2 antibody described herein comprises a heavy chain variable region (V_{H}) and a light chain variable region (V_{L}). The heavy chain variable region (V_{H}) comprises a CDR1 having an amino acid sequence as set forth in SEQ ID NO: 1, a CDR2 having an amino acid sequence as set forth in SEQ ID NO: 2 and/or a CDR3 having an amino acid sequence as set forth in SEQ ID NO: 3, and the light chain variable region (V_{L}) comprises a CDR1 having an amino acid sequence as set forth in SEQ ID NO: 4, a CDR2 having an amino acid sequence as set forth in SEQ ID NO: 5 and/or a CDR3 having an amino acid sequence as set forth in SEQ ID NO: 6. In some embodiments, the anti-CLDN18.2 antibody comprises a heavy chain CDR1 (HCDR1) as set forth in SEQ ID NO: 1, an HCDR2 as set forth in SEQ ID NO: 2 and an HCDR3 as set forth in SEQ ID NO: 3, as well as a light chain CDR1 (LCDR1) as set forth in SEQ ID NO: 4, an LCDR2 as set forth in SEQ ID NO: 5 and an LCDR3 as set forth in SEQ ID NO: 6. In some embodiments, the anti-CLDN18.2 antibody comprises a CDR1, a CDR2 and a CDR3 of the heavy chain variable region as set forth in SEQ ID NO: 7, as well as a CDR1, a CDR2 and a CDR3 of the light chain variable region as set forth in SEQ ID NO: 8.

In some embodiments, the anti-CLDN18.2 antibody described herein comprises a heavy chain variable region (V_{H}) having an amino acid sequence as set forth in SEQ ID NO: 7. In some embodiments, the anti-CLDN18.2 antibody described herein comprises a light chain variable region (V_{L}) having an amino acid sequence as set forth in SEQ ID NO: 8. In some embodiments, the anti-CLDN18.2 antibody described herein comprises a heavy chain variable region (V_{H}) having an amino acid sequence as set forth in SEQ ID NO: 7 and a light chain variable region (V_{L}) having an amino acid sequence as set forth in SEQ ID NO: 8. In some embodiments, the anti-CLDN18.2 antibody comprises a heavy chain variable region as set forth in SEQ ID NO: 7 and a light chain variable region as set forth in SEQ ID NO: 8.

In some embodiments, the anti-CLDN18.2 antibody described herein further comprises an immunoglobulin constant region. In some embodiments, the immunoglobulin constant region comprises a heavy chain constant region and/or a light chain constant region. The heavy chain constant region comprises C_{H1}, C_{H1}-C_{H2} or C_{H1}-C_{H3} regions, and the light chain constant region comprises a C_{L} region.

In some embodiments, the anti-CLDN18.2 antibody described herein comprises a heavy chain having an amino acid sequence as set forth in SEQ ID NO: 9 and a light chain having an amino acid sequence as set forth in SEQ ID NO: 10. In some embodiments, the anti-CLDN18.2 antibody comprises a heavy chain as set forth in SEQ ID NO: 9 and a light chain as set forth in SEQ ID NO: 10.

Exemplary amino acid sequences used in some embodiments are listed in Table A below.

TABLE A
Exemplary amino acid sequences

| SEQ | | |
|---|---|---|
| ID NO. | Amino acid sequence | Name |
| 1 | GYNMN | HCDR1 |
| 2 | NIDPYYGGTSYNQKFKG | HCDR2 |
| 3 | MYHGNAFDY | HCDR3 |
| 4 | KSSQSLLNSGNLKNYLT | LCDR1 |
| 5 | WASTRKS | LCDR2 |
| 6 | QNDYSYPLT | LCDR3 |
| 7 | QVQLVQSGAEVKKPGASVKVSCKASGYTFTG | VH |
| | YNMNWVRQAPGQGLEWMGNIDPYYGGTSY | |
| | NQKFKGRVTMTIDKSTSTVYMELSSLRSEDTA | |
| | VYYCARMYHGNAFDYWGQGTTVTVSS | |
| 8 | DIVMTQSPDSLAVSLGERATINCKSSQSLLNSG | VL |
| | NLKNYLTWYQQKPGQPPKLLIYWASTRKSGV | |
| | PDRFSGSGSGTDFTLTISSLQAEDVAVYYCQN | |
| | DYSYPLTFGGGTKVEIK | |
| 9 | QVQLVQSGAEVKKPGASVKVSCKASGYTFTG | HC |
| | YNMNWVRQAPGQGLEWMGNIDPYYGGTSY | |
| | NQKFKGRVTMTIDKSTSTVYMELSSLRSEDTA | |
| | VYYCARMYHGNAFDYWGQGTTVTVSSSGVH | |
| | TFPAVLQSSGLYSLSSVVTVPSSSLGTQTYICN | |
| | VNHKPSNTKVDKKVEPKSCDKTHTCPPCPAP | |
| | ELLGGPSVFLFPPKPKDTLMISRTPEVTCVVVD | |
| | VSHEDPEVKFNWYVDGVEVHNAKTKPREEQ | |
| | YNSTYRVVSVLTVLHQDWLNGKEYKCKVSN | |
| | KALPAPIEKTISKAKGQPREPQVYTLPPSRDEL | |
| | TKNQVSLTCLVKGFYPSDIAVEWESNGQPEN | |
| | NYKTTPPVLDSDGSFFLYSKLTVDKSRWQQG | |
| | NVFSCSVMHEALHNHYTQKSLSLSPGK | |
| 10 | DIVMTQSPDSLAVSLGERATINCKSSQSLLNSG | LC |
| | NLKNYLTWYQQKPGQPPKLLIYWASTRKSGV | |
| | PDRFSGSGSGTDFTLTISSLQAEDVAVYYCQN | |
| | DYSYPLTFGGGTKVEIKRTVAAPSVFIFPPSDE | |
| | QLKSGTASVVCLLNNFYPREAKVQWKVDNA | |
| | LQSGNSQESVTEQDSKDSTYSLSSTLTLSKAD | |
| | YEKHKVYACEVTHQGLSSPVTKSFNRGEC | |

The present disclosure relates to a pharmaceutical formulation comprising an anti-CLDN18.2 antibody (e.g., the anti-CLDN18.2 antibody described herein). In some embodiments, the concentration of the anti-CLDN18.2 antibody in the pharmaceutical formulation of the present disclosure may be 1 mg/ml-200 mg/mi, 1 mg/ml-190 mg/ml, 10 mg/ml-190 mg/ml, 20 mg/ml-180 mg/ml, 20 mg/ml-170 mg/ml, 20 mg/ml-160 mg/ml, 20 mg/ml-150 mg/ml, 20 mg/ml-140 mg/ml, 20 mg/ml-130 mg/ml, 20 mg/ml-120 mg/ml, 20 mg/ml-110 mg/ml, 20 mg/ml-100 mg/ml, 20 mg/ml-90 mg/ml, 20 mg/ml-80 mg/ml, 20 mg/ml-70 mg/ml, 20 mg/ml-60 mg/ml, 20 mg/ml-50 mg/ml, 20 mg/ml-40 mg/ml or 20 mg/ml-30 mg/ml. In some embodiments, the concentration of the anti-CLDN18.2 antibody is any concentration value in the above-mentioned ranges. For example, as required, the concentration of the anti-CLDN18.2 antibody in the pharmaceutical formulation may be at least 5 mg/ml, at least 10 mg/ml, at least 20 mg/ml, at least 30 mg/ml, at least 40 mg/ml, at least 50 mg/ml, at least 60 mg/ml, at least 70 mg/ml, at least 80 mg/ml, at least 90 mg/ml, at least 100 mg/ml, at least 110 mg/ml, at least 120 mg/ml, at least 130 mg/ml, at least 140 mg/ml, at least 150 mg/ml, at least 160 mg/ml, at least 170 mg/ml, at least 180 mg/ml, at least 190 mg/ml and/or at most 200 mg/ml.

### Buffer

The term “buffer” generally refers to a buffered solution that resists changes in pH by the action of its acid-base conjugate components. The “buffer” used herein refers to a compound solution known to be safe when used in a pharmaceutical formulation and maintains or controls the pH of the formulation in a desired range. Acceptable buffers capable of controlling the pH in a range from a mild acidic pH value to a mild alkaline pH value (e.g., a pH value of 4.5-8.0) include, but are not limited to, one or any combination of a succinic acid buffer, a citric acid buffer, a phosphoric acid buffer, an acetic acid buffer, an arginine buffer, a 2-amino-2-hydroxymethyl-1, 3-propanediol (TRIS) buffer, a histidine buffer, and the like.

The buffer in the formulation solvent may be prepared using any appropriate method known in the art. In some embodiments, the buffer of the present disclosure may be prepared using specific acid-base ion pairs. In one example, excipients of acid-base ion pairs may be accurately weighed and added into pure water that is about 60% of the volume of a target buffer, uniform mixing is conducted, and then the pH value of a resulting solution is determined. When the pH value deviates from a target value, the pH value may be adjusted with appropriate ion pairs. Then, the solution is diluted with pure water to a target weight or a target volume. Finally, the conductivity, osmotic pressure and pH value of the solution are measured for verification.

The stable pharmaceutical formulation of the present disclosure may comprise a buffer such that the pharmaceutical formulation has a pH value of 4.5-8.0, such as a pH value of 4.5-6.0, 6.0-7.0 or 7.0-8.0. In some embodiments, a suitable buffer is used such that the pharmaceutical formulation has a pH value of 4.5-6.0. In particular, the pH value of the pharmaceutical formulation of the present disclosure may be any pH value in the pH ranges listed above, such as 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9 or 8.0.

Examples of the buffer capable of controlling the pH value of the pharmaceutical formulation in a desired range include an acetic acid buffer, a histidine buffer, a citric acid buffer and other organic acid buffers or inorganic acid buffers. Any one of these buffers may be used alone, or 2 or more of these buffers may be combined for use. Preferably, the pharmaceutical formulation of the present disclosure comprises an acetic acid buffer and/or a histidine buffer. More preferably, the pharmaceutical formulation of the present disclosure comprises an acetic acid buffer.

The “acetic acid buffer” refers to a buffer comprising acetate radical ions. The acetic acid buffer may include one or more of acetic acid (e.g., glacial acetic acid), potassium acetate, sodium acetate (e.g., sodium acetate trihydrate), and the like. In some embodiments, the acetic acid buffer is an acetic acid-sodium acetate buffer, such as a glacial acetic acid-sodium acetate trihydrate buffer. In some embodiments, the pH value of the acetic acid buffer may be any pH value in the range of 4.5-6.0, such as 4.5, 4.6, 4.7, 4.8, 4.9, 5.0, 5.1, 5.2, 5.3, 5.4, 5.5, 5.6, 5.7, 5.8, 5.9 or 6.0.

The “histidine buffer” refers to a buffer comprising histidine radical ions. The histidine buffer may include one or more ofhistidine, histidine hydrochloride (e.g., histidine hydrochloride monohydrate), histidine acetate, histidine phosphate, histidine sulfate, and the like. In some embodiments, the histidine buffer may be a histidine-histidine hydrochloride buffer. In some embodiments, the pH value of the histidine buffer may be any pH value in the range of 4.5-6.0, such as 4.5, 4.6, 4.7, 4.8, 4.9, 5.0, 5.1, 5.2, 5.3, 5.4, 5.5, 5.6, 5.7, 5.8, 5.9 or 6.0.

The “citric acid buffer” refers to a buffer comprising citrate radical ions. The citric acid buffer may include one or more of citric acid, monosodium citrate, disodium citrate, trisodium citrate, monopotassium citrate, dipotassium citrate, tripotassium citrate, sodium chloride, potassium chloride, and the like. In some embodiments, the citric acid buffer is a citric acid-trisodium citrate buffer. In some embodiments, the pH value of the citric acid buffer may be any pH value in the range of 4.5-6.0, such as 4.5, 4.6, 4.7, 4.8, 4.9, 5.0, 5.1, 5.2, 5.3, 5.4, 5.5, 5.6, 5.7, 5.8, 5.9 or 6.0.

In some embodiments, the buffer used in the pharmaceutical formulation of the present disclosure is an acetic acid buffer or a histidine buffer, and the pH value of the pharmaceutical formulation of the present disclosure is 4.5-6.0. Without wishing to be bound by any theory, it is believed that the acetic acid buffer and the histidine buffer are better than the citric acid buffer. For example, when the buffer of the pharmaceutical formulation of the present disclosure is an acetic acid buffer or a histidine buffer, the anti-CLDN18.2 antibody in the pharmaceutical formulation is more stable (e.g., more stable at high temperature), the force between antibody molecules is a repulsive force rather than an attractive force, and the risk of aggregation of molecules is lower.

The concentration of the buffer described herein refers to the concentration of buffer ions in the buffer. In some embodiments, the concentration of a suitable buffer used in the pharmaceutical formulation of the present disclosure may be 5 mmol/L-100 mmol/L, 5 mmol/L-90 mmol/L, 5 mmol/L-80 mmol/L, 5 mmol/L-70 mmol/L, 5 mmol/L-60 mmol/L, 5 mmol/L-50 mmol/L, 10 mmol/L-50 mmol/L, 10 mmol/L-40 mmol/L, 10 mmol/L-30 mmol/L and 10 mmol/L-20 mmol/L. In some embodiments, the concentration of the buffer is any concentration value in the above range. For example, according to requirements, the concentration of the buffer may be 5 mmol/L, at least 10 mmol/L, at least 15 mmol/L, at least 20 mmol/L, at least 25 mmol/L, at least 30 mmol/L, at least 35 mmol/L, at least 40 mmol/L, at least 45 mmol/L, at least 50 mmol/L, at least 55 mmol/L, at least 60 mmol/L, at least 65 mmol/L, at least 70 mmol/L, at least 75 mmol/L, at least 80 mmol/L, at least 85 mmol/L, at least 90 mmol/L, at least 95 mmol/L and/or at most 100 mmol/L, depending on the specific buffer and the stability required for the pharmaceutical formulation.

In some embodiments, the buffer used in the pharmaceutical formulation of the present disclosure is an acetic acid buffer, such as an acetic acid-sodium acetate buffer, and the concentration may be 5 mmol/L-100 mmol/L. In some embodiments, the concentration of the acetic acid buffer may be 5 mmol/L-100 mmol/L, 5 mmol/L-90 mmol/L, 5 mmol/L-80 mmol/L, 5 mmol/L-70 mmol/L, 5 mmol/L-60 mmol/L, 5 mmol/L-50 mmol/L, 5 mmol/L-45 mmol/L, 5 mmol/L-40 mmol/L, 5 mmol/L-35 mmol/L, 5 mmol/L-30 mmol/L, 5 mmol/L-25 mmol/L, 5 mmol/L-20 mmol/L, 5 mmol/L-15 mmol/L or 5 mmol/L-10 mmol/L.

In some embodiments, the buffer used in the pharmaceutical formulation of the present disclosure is a histidine buffer, such as a histidine-histidine hydrochloride buffer, and the concentration may be 5 mmol/L-100 mmol/L. In some embodiments, the concentration of the histidine buffer may be 5 mmol/L-100 mmol/L, 5 mmol/L-90 mmol/L, 5 mmol/L-80 mmol/L, 5 mmol/L-70 mmol/L, 5 mmol/L-60 mmol/L, 5 mmol/L-50 mmol/L, 5 mmol/L-45 mmol/L, 5 mmol/L-40 mmol/L, 5 mmol/L-35 mmol/L, 5 mmol/L-30 mmol/L, 5 mmol/L-25 mmol/L, 5 mmol/L-20 mmol/L, 5 mmol/L-15 mmol/L or 5 mmol/L-10 mmol/L.

In some embodiments, the buffer used in the pharmaceutical formulation of the present disclosure is an acetic acid buffer or a histidine buffer, and the concentration is 5 mM-50 mM. In some embodiments, the buffer used in the pharmaceutical formulation of the present disclosure is an acetic acid buffer or a histidine buffer, and the concentration is 10 mM-30 mM. In some embodiments, the buffer used in the pharmaceutical formulation of the present disclosure is an acetic acid buffer or a histidine buffer, and the concentration is about 20 mM. In some embodiments, the buffer used in the pharmaceutical formulation of the present disclosure is an acetic acid buffer, and the concentration is about 20 mM.

### Stabilizer

As used herein, the term “stabilizer” refers to an agent capable of preventing or reducing the chemical and/or physical instability of a protein of interest when bound to the protein. Examples of the stabilizer include sugars, alcohols, acids, salts, polymers, and the like. Examples of the sugars include glucose, sucrose, trehalose, lactose, glucan, and the like. Examples of the alcohols include sorbitol, and the like. Examples of the acids include citric acid, phosphoric acid, tartaric acid, amino acid, ethylenediamine tetraacetic acid, and the like. Examples of the salts include sodium sulfate, sodium glutamate, sodium chloride, potassium chloride, ammonium acetate, and the like. Examples of the polymers include polyethylene glycol, povidone, and the like.

In some embodiments, the stabilizer used in the pharmaceutical formulation of the present disclosure is selected from sugars. In some embodiments, the stabilizer used in the pharmaceutical formulation of the present disclosure is selected from alcohols. In some embodiments, the stabilizer used in the pharmaceutical formulation of the present disclosure is selected from sucrose, trehalose, sorbitol or combinations thereof.

In some embodiments, the concentration of the stabilizer used in the pharmaceutical formulation may be 1% (w/v)-20% (w/v), 1% (w/v)-19% (w/v), 1% (w/v)-18% (w/v), 1% (w/v)-17% (w/v), 1% (w/v)-16% (w/v), 1% (w/v)-15% (w/v), 1% (w/v)-14% (w/v), 1% (w/v)-13% (w/v), 1% (w/v)-12% (w/v), 1% (w/v)-11% (w/v), 1% (w/v)-10% (w/v), 2% (w/v)-10% (w/v), 5% (w/v)-10% (w/v), 2% (w/v)-8% (w/v), 2% (w/v)-7% (w/v), 2% (w/v)-6% (w/v) or 2-5% (w/v), depending on the specific stabilizer and the stability required for the pharmaceutical formulation. In some embodiments, the concentration of the stabilizer is any concentration value in the above range.

In some embodiments, the stabilizer used in the pharmaceutical formulation of the present disclosure is sucrose, and the concentration of the sucrose in the pharmaceutical formulation may be 1% (w/v)-20% (w/v). In some embodiments, the concentration of the sucrose in the pharmaceutical formulation may be 1% (w/v)-10% (w/v), 10% (w/v)-20% (w/v) or 5% (w/v)-15% (w/v). In some embodiments, the concentration of the sucrose in the pharmaceutical formulation is 5% (w/v)-10% (w/v). In some embodiments, the concentration of the sucrose in the pharmaceutical formulation is about 9% (w/v).

In some embodiments, the stabilizer used in the pharmaceutical formulation of the present disclosure is trehalose, and the concentration of the trehalose in the pharmaceutical formulation may be 1% (w/v)-20% (w/v). In some embodiments, the concentration of the trehalose in the pharmaceutical formulation may be 1% (w/v)-10% (w/v), 10% (w/v)-20% (w/v) or 5% (w/v)-15% (w/v). In some embodiments, the concentration of the trehalose in the pharmaceutical formulation is 5% (w/v)-10% (w/v). In some embodiments, the concentration of the trehalose in the pharmaceutical formulation is about 9% (w/v).

In some embodiments, the stabilizer used in the pharmaceutical formulation of the present disclosure is sorbitol, and the concentration of the sorbitol in the pharmaceutical formulation may be 1% (w/v)-20% (w/v). In some embodiments, the concentration of the sorbitol in the pharmaceutical formulation may be 1% (w/v)-10% (w/v), 10% (w/v)-20% (w/v) or 5% (w/v)-15% (w/v). In some embodiments, the concentration of the sorbitol in the pharmaceutical formulation is 2% (w/v)-8% (w/v). In some embodiments, the concentration of the sorbitol in the pharmaceutical formulation is about 5% (w/v).

### Surfactant

As used herein, the term “surfactant” refers to an organic substance with an amphiphilic structure having both hydrophilicity and hydrophobicity; in other words, the organic substance contains groups with opposite solubility trends, such as oil-soluble hydrocarbon chains and water-soluble ionic groups. Depending on charge of surface active moiety, the surfactant may include anionic surfactants, cationic surfactants and nonionic surfactants.

Exemplary surfactant includes polysorbate (e.g., polysorbate 80), poloxamer (e.g., poloxamer 188), Triton, polyethylene glycol, polypropylene glycol and copolymers of ethylene glycol and propylene glycol (e.g., Pluronics, PF68, and the like). In some embodiments, the surfactant used in the pharmaceutical formulation of the present disclosure is selected from polysorbate 80 (also called PS 80 or Tween 80), poloxamer 188, or combinations thereof.

In some embodiments, the concentration of the surfactant used in the pharmaceutical formulation of the present disclosure may be 0.005% (w/v)-0.4% (w/v), 0.01% (w/v)-0.3% (w/v), 0.01% (w/v)-0.2% (w/v), 0.05% (w/v)-0.2% (w/v), 0.05% (w/v)-0.1% (w/v) or 0.01% (w/v)-0.1% (w/v), depending on the specific surfactant and the stability required for the pharmaceutical formulation. In some embodiments, the concentration of the surfactant is any concentration value in the above range.

In some embodiments, the surfactant used in the pharmaceutical formulation of the present disclosure is polysorbate 80, and the concentration of the polysorbate 80 in the pharmaceutical formulation may be 0.005% (w/v)-0.4% (w/v). In some embodiments, the concentration of the polysorbate 80 in the pharmaceutical formulation may be 0.005% (w/v)-0.1% (w/v) or 0.1% (w/v)-0.4% (w/v). In some embodiments, the concentration of the polysorbate 80 in the pharmaceutical formulation may be 0.01% (w/v)-0.1% (w/v). In some embodiments, the concentration of the polysorbate 80 in the pharmaceutical formulation may be about 0.05% (w/v).

In some embodiments, the surfactant used in the pharmaceutical formulation of the present disclosure is poloxamer 188, and the concentration of the poloxamer 188 in the pharmaceutical formulation may be 0.005% (w/v)-0.4% (w/v). In some embodiments, the concentration of the poloxamer 188 in the pharmaceutical formulation may be 0.005% (w/v)-0.1% (w/v) or 0.1% (w/v)-0.4% (w/v). In some embodiments, the concentration of the poloxamer 188 in the pharmaceutical formulation may be 0.05% (w/v)-0.2% (w/v).

### Chelating Agent

As used herein, the term “chelating agent” refers to compounds capable of reacting with certain metal ions (e.g., manganese (II), copper (II), iron (III) and cobalt (II)) to form stable water-soluble metal complexes. Chelation refers to the formation or presence of two or more separate bonds between a ligand and a single central atom. Exemplary chelating agent include, but are not limited to, ethylenediamine tetraacetic acid (EDTA), diethylenetriaminepentaacetic acid (DTPA), N-(1, 2-dicarboxylethyl)-D, L-aspartic acid (IDHA), ethylenediamine-N, N′-bis (EDDHA) and N, N-bis(2-hydroxyphenyl) ethylenediamine-N, N′-diacetic acid (HBED) or salt forms thereof, such as EDTA-2Na and EDTA-4Na. In some embodiments, the chelating agent used in the pharmaceutical formulation of the present disclosure may be a chelating salt of EDTA (e.g., a disodium salt of EDTA, namely EDTA-2Na; or a tetrasodium salt of EDTA, namely EDTA-4Na).

In some embodiments, the concentration of the chelating agent used in the pharmaceutical formulation of the present disclosure may be 30 μmol/L-350 μmol/L, 30 μmol/L-330 μmol/L, 30 μmol/L-300 μmol/L, 30 μmol/L-270 μmol/L, 30 μmol/L-250 μmol/L, 30 mol/L-230 μmol/L, 30 mol/L-200 μmol/L, 30 mol/L-170 mol/L, 30 μmol/L-150 mol/L, 30 μmol/L-130 μmol/L, 40 μmol/L-100 μmol/L, 40 mol/L-90 mol/L, 40 μmol/L-80 μmol/L, 40 μmol/L-70 mol/L, 40 μmol/L-60 μmol/L or 40 μmol/L-50 μmol/L, depending on the specific chelating agent and the stability required for the pharmaceutical formulation. In some embodiments, the concentration of the chelating agent is any concentration value in the above range.

In some embodiments, the chelating agent used in the pharmaceutical formulation of the present disclosure is disodium edetate (EDTA-Na₂), and the concentration is 30 μmol/L-350 μmol/L or 40 μmol/L-60 μmol/L. In some embodiments, the chelating agent used in the pharmaceutical formulation of the present disclosure is disodium edetate (EDTA-Na₂), and the concentration is about 50 μmol/L.

In another aspect, the present disclosure provides use of the chelating agent in preventing the degradation of a surfactant (e.g., polysorbate 80) in the pharmaceutical formulation (e.g., the pharmaceutical formulation provided by the present disclosure).

In another aspect, the present disclosure provides use of the chelating agent in preventing the reduction of the stability of a protein (e.g., an antibody) in the pharmaceutical formulation (e.g., the pharmaceutical formulation provided by the present disclosure).

### Other Materials

In certain embodiments, the pharmaceutical formulation of the present disclosure may further comprise other excipients, for example, but not limited to, an isotonic agent, a diluent, and the like.

The term “isotonic agent” refers to a compound or a composition that provides a drug with an appropriate osmotic tension to prevent net flow of water crossing a cell membrane in contact with the drug. In some embodiments, the pharmaceutical formulation of the present disclosure has the same osmotic pressure as human blood. Suitable isotonic agents include, but are not limited to, glycerol, amino acids or proteins (e.g., glycine or albumin), salts (e.g., sodium chloride) and sugars (e.g., glucose, mannitol, sucrose and lactose).

The term “diluent” refers to pharmaceutically acceptable reagents that may be used for diluting the pharmaceutical formulation of the present disclosure. Typical diluents include water, normal saline, bacteriostatic agents for injection, pH buffers, sterile salt solutions, Ringer's solutions or glucose solutions.

### Formulation

In one aspect, the present disclosure provides a stable pharmaceutical formulation comprising an anti-CLDN18.2 antibody (e.g., the specific anti-CLDN18.2 antibody provided herein), a buffer, a stabilizer and a surfactant. The pharmaceutical formulation has a pH value of 4.5-8.0. In some embodiments, the pH value is 4.5-6.0 to achieve sufficient stability.

In some embodiments, the pharmaceutical formulation of the present disclosure comprises:
i) an anti-CLDN18.2 antibody (e.g., the specific anti-CLDN18.2 antibody provided herein), where the concentration of the anti-CLDN18.2 antibody is 1 mg/ml-200 mg/ml, preferably 20 mg/ml-40 mg/ml, and more preferably about 30 mg/ml;
ii) a buffer, where the buffer is preferably an acetic acid buffer or a histidine buffer, more preferably an acetic acid buffer, and the concentration of the buffer in the pharmaceutical formulation is 5 mmol/L-100 mmol/L, preferably 5 mmol/L-50 mmol/L or 10 mmol/L-30 mmol/L, and more preferably about 20 mmol/L;
iii) a stabilizer, where the stabilizer is preferably sucrose, trehalose or sorbitol,
a) the stabilizer is preferably sucrose or trehalose, and the concentration of the stabilizer in the pharmaceutical formulation is 1% (w/v)-20% (w/v), preferably 1% (w/v)-10% (w/v) or 5% (w/v)-10% (w/v), and more preferably about 9% (w/v), or
b) the stabilizer is preferably sorbitol, and the concentration of the stabilizer in the pharmaceutical formulation is 1% (w/v)-20% (w/v), preferably 1% (w/v)-10% (w/v) or 2% (w/v)-8% (w/v), and more preferably about 5% (w/v);
iv) a surfactant, where the surfactant is preferably polysorbate 80 or poloxamer 188,
a) the surfactant is preferably polysorbate 80, and the concentration of the surfactant in the pharmaceutical formulation is 0.005% (w/v)-0.4% (w/v), preferably 0.01% (w/v)-0.2% (w/v), more preferably 0.01% (w/v)-0.1% (w/v), and more preferably 0.05% (w/v), or
b) the surfactant is preferably poloxamer 188, and the concentration of the surfactant in the pharmaceutical formulation is 0.005% (w/v)-0.4% (w/v), preferably 0.01% (w/v)-0.2% (w/v), and more preferably 0.05% (w/v)-0.2% (w/v); and
v) optionally, a chelating agent, where the chelating agent is preferably EDTA or a salt thereof (e.g., EDTA-2Na), and the concentration of the chelating agent in the pharmaceutical formulation is 30 μM-350 μM or 40 μM-60 μM, and
the pharmaceutical formulation has a pH value of about 4.5-6.0, preferably about 5.0-5.5.

In some embodiments, the pharmaceutical formulation of the present disclosure comprises an anti-CLDN18.2 antibody (e.g., the specific anti-CLDN18.2 antibody provided by the present disclosure) having a concentration of about 1 mg/ml-200 mg/ml and an acetic acid buffer or a histidine buffer having a concentration of about 5 mmol/L-100 mmol/L, where the pH value of the pharmaceutical formulation is 4.5-6.0. In some embodiments, the pharmaceutical formulation comprises an anti-CLDN18.2 antibody having a concentration of about 20 mg/ml-40 mg/ml. In some embodiments, the pharmaceutical formulation comprises an acetic acid buffer or a histidine buffer having a concentration of about 10-30 mmol/L.

In some embodiments, the pharmaceutical formulation of the present disclosure comprises an anti-CLDN18.2 antibody (e.g., the specific anti-CLDN18.2 antibody provided by the present disclosure) having a concentration of about 1 mg/ml-200 mg/ml, an acetic acid buffer or a histidine buffer having a concentration of about 5 mmol/L-100 mmol/L, and sucrose, trehalose or sorbitol having a concentration of about 1% (w/v)-20% (w/v), where the pH value of the pharmaceutical formulation is 4.5-6.0. In some embodiments, the pharmaceutical formulation comprises an anti-CLDN18.2 antibody having a concentration of about 20 mg/ml-40 mg/ml. In some embodiments, the pharmaceutical formulation comprises an acetic acid buffer or a histidine buffer having a concentration of about 10 mmol/L-30 mmol/L. In some embodiments, the pharmaceutical formulation comprises sucrose or trehalose having a concentration of about 5% (w/v)-10% (w/v). In some embodiments, the pharmaceutical formulation comprises sorbitol having a concentration of about 2% (w/v)-8% (w/v).

In some embodiments, the pharmaceutical formulation of the present disclosure comprises an anti-CLDN18.2 antibody (e.g., the specific anti-CLDN18.2 antibody provided by the present disclosure) having a concentration of about 1 mg/ml-200 mg/ml, an acetic acid buffer or a histidine buffer having a concentration of about 5 mmol/L-100 mmol/L, sucrose, trehalose or sorbitol having a concentration of about 1% (w/v)-20% (w/v), and polysorbate 80 or poloxamer 188 having a concentration of about 0.005% (w/v)-0.4% (w/v), where the pH value of the pharmaceutical formulation is 4.5-6.0. In some embodiments, the pharmaceutical formulation comprises an anti-CLDN18.2 antibody having a concentration of about 20 mg/ml-40 mg/ml. In some embodiments, the pharmaceutical formulation comprises an acetic acid buffer or a histidine buffer having a concentration of about 10 mmol/L-30 mmol/L. In some embodiments, the pharmaceutical formulation comprises sucrose or trehalose having a concentration of about 5% (w/v)-10% (w/v), or sorbitol having a concentration of about 2% (w/v)-8% (w/v). In some embodiments, the pharmaceutical formulation comprises polysorbate 80 having a concentration of about 0.01% (w/v)-0.1% (w/v), or poloxamer 188 having a concentration of about 0.05% (w/v)-0.2% (w/v).

In some embodiments, the pharmaceutical formulation of the present disclosure comprises an anti-CLDN18.2 antibody (e.g., the specific anti-CLDN18.2 antibody provided by the present disclosure) having a concentration of about 1 mg/ml-200 mg/ml, an acetic acid buffer or a histidine buffer having a concentration of about 5 mmol/L-100 mmol/L, sucrose, trehalose or sorbitol having a concentration of about 1% (w/v)-20% (w/v), polysorbate 80 or poloxamer 188 having a concentration of about 0.005% (w/v)-0.4% (w/v), and a chelating agent having a concentration of about 30 μmol/L-350 μmol/L, where the pH value of the pharmaceutical formulation is 4.5-6.0. In some embodiments, the pharmaceutical formulation comprises an anti-CLDN18.2 antibody having a concentration of about 20 mg/ml-40 mg/ml. In some embodiments, the pharmaceutical formulation comprises an acetic acid buffer or a histidine buffer having a concentration of about 10 mmol/L-30 mmol/L. In some embodiments, the pharmaceutical formulation comprises sucrose or trehalose having a concentration of about 5% (w/v)-10% (w/v), or sorbitol having a concentration of about 2% (w/v)-8% (w/v). In some embodiments, the pharmaceutical formulation comprises polysorbate 80 having a concentration of about 0.01% (w/v)-0.1% (w/v), or poloxamer 188 having a concentration of about 0.05% (w/v)-0.2% (w/v). In some embodiments, the pharmaceutical formulation comprises a chelating agent (e.g., EDTA and EDTA-2Na) having a concentration of about 40 μmol/L-60 μmol/L. In some other embodiments, the pharmaceutical formulation of the present disclosure comprises substantially no chelating agent.

In some embodiments, the pharmaceutical formulation of the present disclosure comprises an anti-CLDN18.2 antibody having a concentration of about 20 mg/ml-40 mg/ml, an acetic acid buffer having a concentration of about 10 mmol/L-30 mmol/L, sucrose or trehalose having a concentration of about 5% (w/v)-10% (w/v), and polysorbate 80 having a concentration of about 0.01% (w/v)-0.2% (w/v), where the pH value of the pharmaceutical formulation is about 4.5-6.0 or about 5.0-5.5.

In some embodiments, the pharmaceutical formulation of the present disclosure comprises an anti-CLDN18.2 antibody having a concentration of about 30 mg/ml, an acetic acid buffer having a concentration of about 20 mM, sucrose having a concentration of about 9% (w/v), and polysorbate 80 having a concentration of about 0.05% (w/v), where the pH value is about 5.3.

In some embodiments, the pharmaceutical formulation of the present disclosure comprises an anti-CLDN18.2 antibody having a concentration of about 20 mg/ml-40 mg/ml, an acetic acid buffer having a concentration of about 10 mmol/L-30 mmol/L, sucrose or trehalose having a concentration of about 5% (w/v)-10% (w/v), polysorbate 80 having a concentration of about 0.01% (w/v)-0.2% (w/v), and EDTA or EDTA-2Na having a concentration of about 40 μmol/L-60 μmol/L, where the pH value of the pharmaceutical formulation is about 4.5-6.0 or about 5.0-5.5.

In some embodiments, the pharmaceutical formulation of the present disclosure comprises an anti-CLDN18.2 antibody having a concentration of about 30 mg/ml, an acetic acid buffer having a concentration of about 20 mM, sucrose having a concentration of about 9% (w/v), polysorbate 80 having a concentration of about 0.05% (w/v), and EDTA or EDTA-2Na having a concentration of about 50 μmol/L, where the pH value is about 5.3.

In some embodiments, the pharmaceutical formulation of the present disclosure comprises an anti-CLDN18.2 antibody having a concentration of about 20 mg/ml-40 mg/ml, an acetic acid buffer having a concentration of about 10 mmol/L-30 mmol/L, sorbitol having a concentration of about 2% (w/v)-8% (w/v), and polysorbate 80 having a concentration of about 0.01% (w/v)-0.1% (w/v), where the pH value of the pharmaceutical formulation is about 4.5-6.0 or about 5.0-5.5.

In some embodiments, the pharmaceutical formulation of the present disclosure comprises an anti-CLDN18.2 antibody having a concentration of about 20 mg/ml-40 mg/ml, an acetic acid buffer having a concentration of about 10 mmol/L-30 mmol/L, sorbitol having a concentration of about 2% (w/v)-8% (w/v), polysorbate 80 having a concentration of about 0.01% (w/v)-0.1% (w/v), and EDTA having a concentration of about 40 μmol/L-60 μmol/L, where the pH value of the pharmaceutical formulation is about 4.5-6.0 or about 5.0-5.5.

In some embodiments, the pharmaceutical formulation of the present disclosure comprises an anti-CLDN18.2 antibody having a concentration of about 20 mg/ml-40 mg/ml, a histidine buffer having a concentration of about 10 mmol/L-30 mmol/L, sucrose having a concentration of about 6% (w/v)-12% (w/v), and poloxamer 188 having a concentration of about 0.05% (w/v)-0.2% (w/v), where the pH value of the pharmaceutical formulation is about 4.5-6.0 or about 5.0-5.5.

In some embodiments, the pharmaceutical formulation of the present disclosure comprises an anti-CLDN18.2 antibody having a concentration of about 20 mg/ml-40 mg/ml, a histidine buffer having a concentration of about 10 mmol/L-30 mmol/L, sorbitol having a concentration of about 2% (w/v)-8% (w/v), and poloxamer 188 having a concentration of about 0.05% (w/v)-0.2% (w/v), where the pH value of the pharmaceutical formulation is about 4.5-6.0 or about 5.0-5.5.

In some embodiments, the pharmaceutical formulation of the present disclosure comprises an anti-CLDN18.2 antibody having a concentration of about 20 mg/ml-40 mg/ml, a histidine buffer having a concentration of about 10 mmol/L-30 mmol/L, sucrose having a concentration of about 6% (w/v)-12% (w/v), and polysorbate 80 having a concentration of about 0.01% (w/v)-0.1% (w/v), where the pH value of the pharmaceutical formulation is about 4.5-6.0 or about 5.0-5.5.

In some embodiments, the pharmaceutical formulation of the present disclosure comprises an anti-CLDN18.2 antibody having a concentration of about 20 mg/ml-40 mg/ml, a histidine buffer having a concentration of about 10 mmol/L-30 mmol/L, sorbitol having a concentration of about 2% (w/v)-8% (w/v), and polysorbate 80 having a concentration of about 0.01% (w/v)-0.1% (w/v), where the pH value of the pharmaceutical formulation is about 4.5-6.0 or about 5.0-5.5.

In some embodiments, the pharmaceutical formulation of the present disclosure comprises an anti-CLDN18.2 antibody having a concentration of about 20 mg/ml-40 mg/ml, a histidine buffer having a concentration of about 10 mmol/L-30 mmol/L, sucrose or trehalose having a concentration of about 6% (w/v)-12% (w/v), poloxamer 188 having a concentration of about 0.05% (w/v)-0.2% (w/v), and EDTA having a concentration of about 40 μmol/L-60 μmol/L, where the pH value of the pharmaceutical formulation is about 4.5-6.0 or about 5.0-5.5.

The polymer formation due to chemical degradation or aggregation of antibody molecules, or the deglycosylation, glycosylation modification, oxidation of antibody molecules, or other structural modifications that may reduce at least one functional activity of monomer proteins may result in instability of antibody formulations. As for the pharmaceutical formulation comprising an anti-CLDN18.2 antibody, the anti-CLDN18.2 antibody may be chemically degraded during storage of the pharmaceutical formulation, leading to decrease in the concentration of the antibody. The anti-CLDN18.2 antibody may also aggregate to form polymers that are sometimes insoluble in the form of polymeric molecules comprising multiple antibody molecules, leading to decrease in the content of monomers containing single antibody molecules. Therefore, the increase in the content of polymer antibodies will lead to decrease in the purity of monomer antibodies. Moreover, the turbidity of the pharmaceutical formulation may be increased due to the formation of insoluble polymers.

In some embodiments, the pharmaceutical formulation comprising an anti-CLDN18.2 antibody of the present disclosure may still maintain stability after long-term storage, treatment (e.g., storage) at high temperature and/or multiple freezing and thawing cycles, wherein the physical and/or chemical stability and/or the functional activity and the like of the anti-CLDN18.2 antibody remain relatively constant over the time. In some embodiments, the antibody protein concentration, the protein purity, the protein activity, the pH value of the formulation, the osmotic pressure of the formulation, the appearance of the formulation, insoluble particles in the formulation and the like may be used as indicators of the stability of the pharmaceutical formulation. A variety of analytical technologies for determining the stability of proteins are available in the art, which are described in Peptide and Protein Drug Delivery, 247-301, edited by Vincent Lee, Marcel Dekker Inc., New York, New York Press (1991) and Jones, A. Adv. Drug Delivery Rev. 10: 29-90 (1993).

In some embodiments, the stability of the pharmaceutical formulation may be determined by methods known in the art under selected conditions over a selected period of time. Exemplary methods include, but are not limited to, dynamic light scattering (DLS), size-exclusion chromatography (SEC), cation exchange chromatography (CEX), non-reduced capillary electrophoresis (NR CE-SDS), pH value determination, protein concentration (Protein Conc.) determination and visual inspection.

As shown in examples of the present disclosure, the pharmaceutical formulation comprising an anti-CLDN18.2 antibody provided herein has high stability by visual inspection, such as high stability under long-term storage, high stability at high temperature (e.g., 40° C.) and high stability after freezing and thawing.

In some embodiments, the stable pharmaceutical formulation refers that: in a DLS test for the pharmaceutical formulation, the protein particle size does not change significantly during storage; the KD value is positive, that is to say, the action force between single antibody molecules is a repulsive force, and no aggregation of the single antibody molecules occurs. As shown in examples of the present disclosure, the pharmaceutical formulation comprising the anti-CLDN18.2 antibody provided herein has high stability as shown in a DLS test, such as high stability under long-term storage, high stability at high temperature (e.g., 40° C.) and high stability after freezing and thawing.

In some embodiments, the stable pharmaceutical formulation refers that: in an SEC test for the pharmaceutical formulation, only a small amount of proteins are degraded during storage of the pharmaceutical formulation, and the contents of high polymers or oligomers increase slowly. As shown in examples of the present disclosure, the pharmaceutical formulation comprising the anti-CLDN18.2 antibody provided herein has high stability as shown in an SEC test, such as high stability under long-term storage, high stability at high temperature (e.g., 40° C.) and high stability after freezing and thawing.

In some embodiments, the stable pharmaceutical formulation refers that: in a CEX test for the pharmaceutical formulation, the charge heterogeneity of the pharmaceutical formulation does not change significantly during storage, and the separation degree of acidic peaks and alkaline peaks does not change significantly. As shown in examples of the present disclosure, the pharmaceutical formulation comprising the anti-CLDN18.2 antibody provided herein has high stability as shown in a CEX test, such as high stability under long-term storage, high stability at high temperature (e.g., 40° C.) and high stability after freezing and thawing.

In some embodiments, the stable pharmaceutical formulation refers that: in an NR CE-SDS test for the pharmaceutical formulation, only a small amount of proteins are degraded during storage of the pharmaceutical formulation, and the contents of high polymers or oligomers increase slowly. As shown in examples of the present disclosure, the pharmaceutical formulation comprising the anti-CLDN18.2 antibody provided herein has high stability in an NR CE-SDS test, such as high stability under long-term storage, high stability at high temperature (e.g., 40° C.) and high stability after freezing and thawing.

In some embodiments, the stable pharmaceutical formulation refers that: in a visual inspection test for the pharmaceutical formulation, no obvious changes in the appearance of the pharmaceutical formulation during storage are observed, and the pharmaceutical formulation remains as a clear and colorless liquid.

In some embodiments, the stable pharmaceutical formulation refers that: in a protein concentration test for the pharmaceutical formulation, the change of the protein concentration in the pharmaceutical formulation is no more than +/−20%, no more than +/−19%, no more than +/−18%, no more than +/−17%, no more than +/−16%, no more than +/−15%, no more than +/−14%, no more than +/−13%, no more than +/−12%, no more than +/−11%, no more than +/−10%, no more than +/−9%, no more than +/−8%, no more than +/−7%, no more than +/−6%, no more than +/−5%, no more than +/−4%, no more than +/−3%, no more than +/−2%, no more than +/−1% or no more than +/−0.5%, wherein the protein concentration can be determined by an ultraviolet-visible spectrophotometry in accordance with General Rule 0401 of Chinese Pharmacopoeia (2010 edition), Volume III.

### Preparation of Formulations

In another aspect, the present disclosure provides a method for preparing a pharmaceutical formulation, comprising:
1) providing a formulation solvent and an anti-CLDN18.2 antibody stock solution, wherein the formulation solvent comprises a buffer, a stabilizer and optionally a surfactant; and
2) subjecting the anti-CLDN18.2 antibody stock solution to solvent exchange with the formulation solvent to obtain the pharmaceutical formulation described herein. In some embodiments, the solvent exchange refers to buffer replacement, such as buffer replacement by a dialysis method. In some embodiments, the solvent exchange refers to solvent exchange by filtration. In some embodiments, the filtration refers to ultrafiltration, infiltration, gel filtration and/or other filtration methods well known to persons skilled in the art.

The present disclosure further provides a method for preparing a pharmaceutical formulation, comprising:
1) providing a formulation solvent and an anti-CLDN18.2 antibody stock solution, where the formulation solvent comprises a buffer and a stabilizer, and the anti-CLDN18.2 antibody stock solution comprises a chelating agent (e.g., EDTA and EDTA-2Na);
2) subjecting the anti-CLDN18.2 antibody stock solution to solvent exchange with the formulation solvent to obtain a product, where the product obtained after the solvent exchange comprises substantially no chelating agent; and
3) adding a surfactant to the product obtained after the solvent exchange (i.e., the formulation solvent comprising the anti-CLDN18.2 antibody) to obtain the pharmaceutical formulation described herein.

In some embodiments, the surfactant comprises polysorbate 80.

In some embodiments, the buffer in the formulation solvent is an acetic acid buffer having a concentration of about 20 mM, the stabilizer is sucrose having a concentration of about 9% (w/v), and the surfactant is polysorbate 80 having a concentration of about 0.05% (w/v).

In some embodiments, the solvent exchange refers to buffer replacement. In some embodiments, the anti-CLDN18.2 antibody stock solution is subjected to solvent exchange by dialysis with the formulation solvent. For example, a certain volume of a sample may be placed in a dialysis bag (e.g., Snake Skin® dialysis bag), the dialysis bag is sealed and placed in a target buffer with a volume equal to or greater than 100 times, and stirring is conducted continuously to promote replacement. Dialysis is conducted for an appropriate number of times (e.g., 3 times) for an appropriate period of time (e.g., 4 hours, 4 hours and overnight) under stirring at an appropriate rate (e.g., 150 rpm).

In some embodiments, the solvent exchange refers to liquid exchange by filtration. In some embodiments, the filtration refers to ultrafiltration, infiltration, gel filtration and/or other filtration methods well known to a person skilled in the art.

As used herein, the term “ultrafiltration” refers to a process of separating different substances (e.g., a solvent and a solute) in a mixture (e.g., an anti-CLDN18.2 antibody stock solution) by moving through a membrane (e.g., an ultrafiltration membrane) with different rates in response to a provided pressure driving force.

As used herein, the term “infiltration” refers to a process of separating components in a mixture (e.g., an anti-CLDN18.2 antibody stock solution) based on the molecular sizes of the components in the mixture by a filter, such as a permeable membrane. Usually, a protein stock solution is infiltrated with a membrane that retains a protein and allows a buffer to be exchanged. Thus, a protein-containing stock solution is replaced by a new buffer over the time.

As used herein, the term “gel filtration” refers to a process of using a gel to separate larger molecules from smaller molecules by repelling the molecules (e.g., proteins) that are larger than resin pores so that they pass through a solid phase more quickly than the smaller molecules that are diffused into the resin pores and are thus retained and move slowly through the solid phase.

In another aspect, the present disclosure provides a method for preparing a pharmaceutical formulation, comprising: mixing an anti-CLDN18.2 antibody stock solution of a high concentration with formulation excipients (e.g., a buffer, a stabilizer and optionally a surfactant) of a high concentration, followed by dilution to a target concentration.

### Application

In another aspect, the present disclosure further provides a method for treating diseases in a subject in need thereof, comprising administrating a therapeutically effective amount of the pharmaceutical formulation provided herein to the subject, where the subject has or is suspicious of having diseases that require treatment with an antibody against CLDN18.2.

As used herein, the term “treatment” or “treating” refers to reducing or relieving disease conditions or the severity and/or duration of one or more of the symptoms thereof, inhibiting or preventing the progression of the disease conditions, reducing or ending symptoms associated with a condition, and inhibiting or preventing the recurrence, development, onset or progression of one or more of the symptoms associated with the disease conditions. The subjects in need thereof include subjects having had the diseases.

The term “therapeutically effective amount” refers to a measurable minimum concentration required for treating (e.g., improving or preventing) a particular disease condition.

The pharmaceutical formulation of the present disclosure can be used for treating CLDN18.2 related diseases, such as chronic diseases and acute diseases. The CLDN18.2 related diseases include cancers, and the like. In some embodiments, the CLDN18.2 related diseases refer to cancers expressing CLDN18.2. Examples of the cancers expressing CLDN18.2 include, but are not limited to, lung cancer (e.g., small cell lung cancer, non-small cell lung cancer (NSCLC), lung adenocarcinoma or lung squamous cell carcinoma), gastric cancer (e.g., gastrointestinal cancer), pancreatic cancer, esophageal cancer, liver cancer (e.g., hepatocellular carcinoma/hepatocellular tumor), squamous cell carcinoma, peritoneal carcinoma, brain tumor (e.g., glioblastoma/glioblastoma multiforme (GBM), non-glioblastoma brain tumor or meninges tumor), glioma (e.g., ependymoma, astrocytoma, anaplastic astrocytoma, oligodendroglioma or mixed glioma, such as oligodendroastrocytoma), cervical cancer, ovarian cancer, liver cancer (e.g., hepatoblastoma, hepatocellular carcinoma/hepatocellular tumor and liver cancer), bladder cancer (e.g., uroepithelial carcinoma), breast cancer, colon cancer, colorectal cancer, rectal cancer, endometrial cancer or uterine cancer, salivary adenocarcinoma, kidney cancer (e.g., renal rhabdomyoma), prostate cancer, vulvar cancer, penile cancer, anal cancer (e.g., squamous cell carcinoma of anus), thyroid cancer, head and neck cancer (e.g., nasopharyngeal carcinoma), skin cancer (e.g., melanoma or squamous cell carcinoma), osteosarcoma, Ewing's sarcoma, chondrosarcoma, soft tissue sarcoma (e.g., rhabdomyosarcoma, fibrosarcoma and Kaposi's sarcoma), carcinoid, ocular carcinoma (e.g., retinoblastoma), mesothelioma, lymphocytic/lymphoblastic leukaemia (e.g., acute lymphocytic/lymphoblastic leukemia (ALL) and chronic lymphocytic/lymphoblastic leukemia (CLL) of T cell lineage and B cell precursor lineage), acute myeloid/myeloblastic leukemia (AML) including mast cell leukemia, chronic myeloid/myelocytic/myeloblastic leukemia (CML), hair cell leukemia (HCL) Hodgkin's disease, non-Hodgkin's lymphoma, chronic myeloid monocytic leukemia (CMML), follicular lymphoma (FL), diffuse large B-cell lymphoma (DLCL), mantle cell lymphoma (MCL), Burkett's lymphoma (BL), mycosis fungoides, Cezari syndrome, cutaneous T-cell lymphoma, mast cell tumor, medulloblastoma, renal blastoma, isolated plasma cell tumor, myelodysplastic syndrome, chronic and non-chronic myelodysplastic disease, central nervous system tumor, pituitary adenoma, vestibular schwannoma, primitive neuroectodermal tumor, ependymoma, chorioplexus papilloma, polycythemia vera, thrombocythemia, gallbladder cancer, idiopathic myelofibrosis, and pediatric cancers such as pediatric sarcomas (e.g., neuroblastoma, rhabdomyosarcoma, and osteosarcoma).

The pharmaceutical formulation of the present disclosure can be administered to the subjects by any suitable routes. For example, the pharmaceutical formulation can be administered intravenously to a subject.

In another aspect, the present disclosure provides use of the pharmaceutical formulation in the manufacture of a medicament for preventing and/or treating CLDN18.2 related diseases.

### EXAMPLES

### Summary of Experimental Methods:

In the following examples, a formulation screening experiment was carried out on formulations of the anti-CLDN18.2 antibody. The anti-CLDN18.2 antibody used in the following examples comprises a heavy chain variable region as set forth in SEQ ID NO: 7 and a light chain variable region as set forth in SEQ ID NO: 8. The anti-CLDN18.2 antibody used in the following examples comprises a heavy chain as set forth in SEQ ID NO: 9 and a light chain as set forth in SEQ ID NO: 10.

Formulations of the anti-CLDN18.2 antibody tested in the following examples of the present disclosure were prepared by the following methods:

1. Preparation of a target formulation buffer: a buffer was prepared using specific acid-base ion pairs. Acid-base ion pair excipients were accurately weighed and added into Milli-Q water that was about 60% of the volume of a target buffer, uniform mixing was conducted, and then the pH value of a resulting solution was determined. Ifthe pH value deviated from a target value, the pH value may be adjusted with appropriate ion pairs. Then, the solution was diluted with Milli-Q water to a target weight or a target volume. Finally, the conductivity, osmotic pressure, and pH value of the solution were measured for verification.

2. Preparation of samples (by a dialysis method or a direct dilution method):

a) Dialysis method: a buffer comprising the anti-CLDN18.2 antibody (namely stock solution, also abbreviated as DS in the following examples) was exchanged into a target formulation buffer by a dialysis method. Specifically, a certain volume of a DS sample was placed in a dialysis bag (e.g., Snake Skin® dialysis bag), which was sealed and placed in a target buffer with a volume equal to or greater than 100 times, and stirring was conducted continuously to promote the exchange. Dialysis was performed for three times, i.e., respectively for 4 hours, for 4 hours, and for overnight, and the stirring rate was 150 rpm.

b) Direct dilution method: an excipient stock solution of a high concentration and a surfactant stock solution of a high concentration were added into DS of a high concentration, and then the DS was diluted to a target concentration with a target buffer system.

The formulations of the anti-CLDN18.2 antibody tested in the following examples of the present disclosure were analyzed by the following analytical methods:

1. Dynamic light scattering (DLS): the protein particle size and the distribution were determined by dynamic light scattering (DLS), and parameters of the method were as follows: collection was conducted for 5 seconds for a total of 20 times for each measurement, and measurement was conducted at a temperature of 25° C.

2. Size-exclusion chromatography (SEC): the protein aggregation was determined by an SEC method with an Agilent 1260 system and a TSKgel G3000SWXL column (300×7.8 mm, 5 μm). A mobile phase included a 50 mM sodium phosphate buffer and 300 mM NaCl, and the pH value was 6.8+0.1. The flow rate was 1.0 mL/min. A sample was diluted to 10 mg/mL and detected at a volume of 10 μL and a wavelength of 280 nm.

3. Cation exchange chromatography (CEX): the charge heterogeneity of proteins was determined by CEX with a Thermo Propac Elite WCX-10 4 mm×150 mm 5 um column in an Agilent 1260 Infinity system. A sample was diluted to 2.00 mg/mL with a mixed solution of a mobile phase A and a mobile phase B.

4. Non-reduced capillary electrophoresis (NR CE-SDS): protein fragments were determined by a CE-SDS (NR) method. A standard sample or a test sample was diluted to 4 mg/mL with a phosphoric acid-citric acid buffer, and then 25 μL of the sample was subjected to vortex mixing with 75 μL of an SDS sample buffer and 5 μL of NEM (100 mM N-ethylmaleimide), followed by denaturation treatment. The denatured sample was subjected to centrifugation, followed by incubation at 70+2° C. for 10+2 minutes, cooling at room temperature and centrifugation again. Separation was performed on PA800 plus by using an SDS separation gel kit and an uncoated fused quartz capillary tube.

5. pH: the pH of a sample was measured by using a Seven Compact pH meter equipped with an Inlab® ExpertPro electrode.

6. Protein Concentration (Protein Conc.): the absorbance was determined by a Nano Drop 2000 spectrophotometer at 280 nm to obtain the protein concentration. The extinction coefficient (E1%) used throughout the study was 1.511 L/g-cm. Each sample was measured for twice at a loading volume of 2.0 μL. The average concentration was reported.

7. Visual inspection: the appearance of a sample was inspected by a YB-2 clarity detector under a black background. The transparency and the color were reported.

Sources of reagents used in the experiments are as follows, and other reagents are conventional reagents in the art.

| Reagent name | Supplier | Item number |
|---|---|---|
| Citric acid monohydrate | Merck | 137003.1000 |
| Trisodium citrate dihydrate | Merck | 1.06446.1000 |
| Histidine | Merck | 1.04352.1000 |
| Histidine hydrochloride | Merck | 1.04354.0500 |
| monohydrate | | |
| Sodium acetate trihydrate | Merck | 1.37012.1000 |
| Glacial acetic acid | J.T Baker | UN2789 |
| Polysorbate 80 | NOF | Polysorbate80(HX2) |
| Sucrose | Pfanstichl | S-124-2-MC |
| Arginine hydrochloride | Merck | 1.01587.1000 |
| Sodium chloride | Merck | 1.16224.5000 |
| Trehalose dihydrate | Pfanstiebl | T-104-4 |
| Sorbitol | MERCK | 1.11597.9028 |
| Poloxamer 188 | MERCK | 1.37065.1000 |
| PVDF membrane | Merck Millipore | 33 mm, 0.22 μm |
| PVDF membrane | Merck Millipore | 13 mm, 0.22 μm |
| PES membrane | Merck Millipore | 33 mm, 0.22 μm |

### Example 1: pH Screening Experiment

### 1. Experimental Design

A pH screening experiment was carried out on formulations of the anti-CLDN18.2 antibody. The anti-CLDN18.2 antibody protein was exchanged into a formulation buffer as shown in Table 1 by dialysis and then diluted to 30.0 mg/mL with a corresponding formulation buffer. Finally, the samples were filtered with a 0.22 μm PVDF membrane and respectively loaded into a 2 mL vial packaging container system by a pipette. The samples were incubated under different conditions listed in Table 1 and then tested.

TABLE 1
Design of a pH screening experiment

| | Protein | | |
|---|---|---|---|
| | concen- | | Stability conditions |
| Formula | tration | Description | and test items |
| F1 | 30.0 | 20 mM acetate, pH 4.5 | An X test and a Y test |
| F2 | mg/mL | 20 mM acetate, pH 5.0 | described in the note |
| F3 | | 20 mM acetate, pH 5.5 | column were carried out |
| F4 | | 20 mM histidine, pH 5.0 | on the 0 day, and after |
| F5 | | 20 mM histidine, pH 5.5 | incubation was |
| F6 | | 20 mM histidine, pH 6.0 | performed at 40° C. for |
| F7 | | 20 mM citrate, pH 5.5 | 14 days, the X test |
| F8 | | 20 mM citrate, pH 6.0 | described in the note |
| F9 | | 20 mM citrate, pH 6.5 | column was carried out |
| | | | on the 14^{th} day (T0^{X Y} |
| | | | 40° C. (14 D)X). |

Note:
1. ^{X}= Conc., SEC, CEX, DLS, NR CE-SDS, visual inspection, and ^{Y}= pH.
2. Considering that the KD values of the formulations F7, F8 and F9 were negative, and that these formulations were slightly opalescent in appearance and had a large radius of single antibody molecules at T0, it is believed that the stability of these formulations was obviously lower than that of other formulations. Thus, other tests were not carried out on the F7, F8 and F9.

### 2. Analysis of Results

(1) At T0, the Formulations F7, F8 and F9 were slightly opalescent and colorless liquids in appearance, and other formulations were clear and colorless liquids. After incubation was performed at 40° C. for 14 days, no significant changes in the appearance of all the other formulations were observed except for the Formulations F7, F8 and F9.

TABLE 2
Appearance, pH and concentration results
of a pH screening experiment at 40° C.

Appearance

| Formula | T0 | 14 D |
|---|---|---|
| F1 | CC | CC |
| F2 | CC | CC |
| F3 | CC | CC |
| F4 | CC | CC |
| F5 | CC | CC |
| F6 | CC | CC |
| F7 | SOC | NT |
| F8 | SOC | NT |
| F9 | SOC | NT |

Note:
CC refers to clear and colorless, SOC refers to slightly opalescent and colorless, and NT refers to not test.

(2) At T0, the KD values of the Formulations F7, F8 and F9 were negative, indicating that the action force between single antibody molecules was an attractive force. The KD values of the other formulas were positive, indicating that the action force between single antibody molecules was a repulsive force. At T0, the radius of single antibody molecules in the Formulations F7, F8 and F9 was obviously larger than that of other formulations. Thus, the Formulations F1, F2, F3, F4, F5 and F6 all meet the requirements for the KD value and the particle size at T0.

TABLE 3
DLS results of a pH screening experiment at 40° C.

T0

| Formula | KD (mL/g) | Particle size | Polydispersity index % |
|---|---|---|---|
| F1 | 65.7 | 2.0 | 0.0 |
| F2 | 32.2 | 3.0 | 0.7 |
| F3 | 20.5 | 3.5 | 0.2 |
| F4 | 19.5 | 3.5 | 0.4 |
| F5 | 18.3 | 3.6 | 0.4 |
| F6 | 23.3 | 3.3 | 0.9 |
| F7 | −7.79 | 6.9 | 2.9 |
| F8 | −7.94 | 7.6 | 6.8 |
| F9 | −8.15 | 7.2 | 3.7 |

(3) An SEC test was carried out on the Formulations F1, F2, F3, F4, F5 and F6. As shown in Table 4, after incubation was performed at 40° C. for 14 days, the Formulations F1, F2, F3, F4, F5 and F6 had fragments (LMW) formed by degradation of a small amount of main peaks (MPs) and slow increase of high polymers (HMW). Thus, the acceptance criteria under such test conditions was satisfied, and all the formulations meet the requirements for stability.

TABLE 4
SEC results of a pH screening experiment at 40° C.

| | T0 | | | 40° C. for 14 days | | |
|---|---|---|---|---|---|---|
| | MP | HMW | LMW | MP | HMW | LMW |
| Formula | % | % | % | % | % | % |
| F1 | 99.6 | 0.3 | 0.1 | 97.0 | 0.3 | 2.7 |
| F2 | 99.5 | 0.4 | 0.2 | 96.9 | 0.5 | 2.6 |
| F3 | 99.5 | 0.4 | 0.1 | 96.7 | 0.8 | 2.5 |
| F4 | 99.6 | 0.3 | 0.1 | 97.0 | 0.4 | 2.5 |
| F5 | 99.5 | 0.4 | 0.1 | 97.3 | 0.4 | 2.3 |
| F6 | 99.5 | 0.4 | 0.1 | 97.3 | 0.5 | 2.2 |

(4) A CEX test was performed. After incubation was performed at 40° C. for 14 days, all the formulations had the degradation of main peaks and the formation of acidic peaks. Thus, the acceptance criteria under such test conditions was satisfied. The formulations had no obvious differences.

TABLE 5
CEX results of a pH screening experiment at 40° C.

| | T0 | | | 40° C. for 14 days | | |
|---|---|---|---|---|---|---|
| | | Acidic | Alkaline | | Acidic | Alkaline |
| Formula | MP % | peak % | peak % | MP % | peak % | peak % |
| F1 | 61.9 | 22.3 | 15.8 | 51.8 | 35.4 | 12.8 |
| F2 | 62.0 | 22.3 | 15.7 | 52.6 | 35.3 | 12.1 |
| F3 | 62.0 | 22.4 | 15.6 | 53.7 | 34.6 | 11.8 |
| F4 | 62.2 | 22.4 | 15.4 | 52.5 | 32.5 | 15.0 |
| F5 | 62.2 | 22.5 | 15.3 | 54.1 | 32.5 | 13.5 |
| F6 | 62.1 | 22.7 | 15.2 | 55.4 | 32.8 | 11.8 |

(5) An NR CE-SDS test was carried out. After incubation was performed at 40° C. for 14 days, all the formulations had the degradation of a small amount of main peaks and the formation of fragments. Thus, the acceptance criteria under such test conditions was satisfied.

TABLE 6
NR CE-SDS results of a pH screening experiment at 40° C.

| | T0 | | | 40° C. for 14 days | | |
|---|---|---|---|---|---|---|
| | MP | HMW | LMW | MP | HMW | LMW |
| Formula | % | % | % | % | % | % |
| F1 | 97.2 | 0.1 | 2.7 | 94.3 | 0.4 | 5.3 |
| F2 | 97.1 | 0.2 | 2.7 | 94.8 | 0.5 | 4.8 |
| F3 | 96.9 | 0.3 | 2.8 | 94.5 | 0.6 | 4.9 |
| F4 | 97.0 | 0.3 | 2.7 | 94.8 | 0.4 | 4.8 |
| F5 | 97.0 | 0.3 | 2.7 | 95.5 | 0.3 | 4.2 |
| F6 | 97.1 | 0.2 | 2.6 | 95.3 | 0.3 | 4.3 |

In conclusion, after incubation was performed at 40° C., the citric acid system was excluded based on the DLS and appearance results, because the action force between single antibody molecules was an attractive force and the single antibody molecules had a large radius, indicating that the single antibody molecules had the risk of aggregation in long-term storage. Meanwhile, the formulations (namely Formulations F1, F2, F3, F4, F5 and F6) in the acetic acid system and the histidine system were relatively stable, such that the stability requirements for protein formulations could be met.

### Example 2: Surfactant Screening Experiment-1

### 1. Experimental Design

A surfactant screening experiment was carried out on the anti-CLDN18.2 antibody protein. The anti-CLDN18.2 antibody protein was filtered with a 0.22 μm PVDF membrane, a sucrose solution and a polysorbate 80 solution were added, and then the anti-CLDN18.2 antibody protein was diluted with a 20 mM acetic acid buffer with a pH value of 5.5 and milli-Q water and prepared into the following formulations (F10, F11, F12 and F13). Acetic acid and sucrose were used as a buffer and a stabilizer, respectively. The final concentration of the anti-CLDN18.2 antibody protein was 30.0 mg/mL. Finally, samples were filtered with a 0.22 μm PVDF membrane and respectively loaded into a 2 mL vial packaging container system by a pipette, followed by stability studies listed in Table 7. The samples were tested after incubation.

TABLE 7
Design of a surfactant screening experiment

| | | | | Surfactant | |
|---|---|---|---|---|---|
| | Protein | Buffer | Surfactant | concentration | Stability conditions and |
| Formula | concentration | system | type | (W/V) | test items |
| F10 | 30.0 mg/mL | 20 mM | Polysorbate | 0.01% | An X test and a Y test |
| F11 | | acetate and | 80 | 0.02% | described in the note |
| F12 | | 9% (w/v) | | 0.05% | column were carried out |
| F13 | | of sucrose, | | 0.1% | on the 0 day, and after |
| | | pH 5.5 | | | incubation was |
| | | | | | performed at 40° C. for 7 |
| | | | | | or 14 days, the X test |
| | | | | | described in the note |
| | | | | | column was carried out |
| | | | | | on the 7^{th} day and the 14^{th} |
| | | | | | day separately (T0^{X Y} |
| | | | | | 40° C. (7 D, 14 D)^{X}). |
| | | | | | After stirring was |
| | | | | | conducted at a rate of 200 |
| | | | | | rpm, the X test described |
| | | | | | in the note column was |
| | | | | | carried out on the 5^{th} day |
| | | | | | and the 7^{th} day separately |
| | | | | | (200 rpm: 5 D, 7 D)^{X}. |

Note:
^{X}= Conc., SEC, CEX, DLS, NR CE-SDS, visual inspection; ^{Y}= pH.

### 2. Analysis of Results

According to the 40° C. data (as shown in Table 8) and the vibration data (as shown in Table 9), it indicated that the polysorbate 80 having the concentration range of 0.01% (w/v)-0.1% (w/v) had an obvious protection effect on the stability of single antibody molecules. All the formulations meet the test requirements, and the polysorbate 80 having a concentration of 0.05% (w/v) was used for further evaluation in subsequent experiments.

TABLE 8
Results of a surfactant screening experiment at 40° C.

| | | Formulation | | | | | | | | | | | |
|---|---|---|---|---|---|---|---|---|---|---|---|---|---|
| | | F10 | | | F11 | | | F12 | | | F13 | | |
| Duration day at 40° C. | | 0 | 7 | 14 | 0 | 7 | 14 | 0 | 7 | 14 | 0 | 7 | 14 |
| Appearance | | CC | CC | CC | CC | CC | CC | CC | CC | CC | CC | CC | CC |
| Concentration (mg/mL) | | 30.2 | 30.0 | 30.2 | 30.3 | 30.2 | 30.3 | 30.2 | 30.2 | 30.6 | 30.1 | 30.1 | 30.5 |
| pH | | 5.4 | NA | NA | 5.4 | NA | NA | 5.4 | NA | NA | 5.5 | NA | NA |
| DLS | Particle size (nm) | 4.4 | 4.3 | 4.3 | 4.4 | 4.4 | 4.4 | 4.3 | 4.3 | 4.3 | 4.3 | 4.3 | 4.3 |
| | Polydispersity index % | 5.6 | 5.3 | 6.2 | 5.8 | 4.9 | 4.8 | 5.7 | 5.1 | 5.7 | 4.7 | 4.7 | 4.2 |
| SEC | HMW % | 0.4 | 0.6 | 0.7 | 0.4 | 0.6 | 0.8 | 0.4 | 0.7 | 0.9 | 0.4 | 0.7 | 1.0 |
| | MP % | 99.5 | 99.1 | 96.6 | 99.5 | 99.1 | 96.4 | 99.5 | 99.0 | 96.3 | 99.5 | 99.0 | 96.4 |
| | LMW % | 0.1 | 0.3 | 2.7 | 0.1 | 0.3 | 2.8 | 0.1 | 0.3 | 2.8 | 0.1 | 0.3 | 2.7 |
| NR | HMW % | 0.2 | 0.4 | 0.5 | 0.3 | 0.3 | 0.6 | 0.2 | 0.4 | 0.5 | 0.2 | 0.3 | 0.6 |
| CE-SDS | MP % | 97.1 | 95.7 | 95.0 | 97.0 | 95.7 | 94.5 | 96.8 | 95.7 | 94.8 | 97.0 | 95.9 | 94.4 |
| | LMW % | 2.6 | 4.0 | 4.5 | 2.7 | 4.0 | 4.8 | 3.0 | 3.9 | 4.7 | 2.7 | 3.8 | 4.9 |
| CEX | Acidic peak % | 22.8 | 27.8 | 36.0 | 22.8 | 27.9 | 36.6 | 22.9 | 28.0 | 36.9 | 23.0 | 28.1 | 37.5 |
| | MP % | 62.1 | 58.4 | 52.4 | 62.1 | 58.3 | 51.9 | 62.1 | 58.1 | 51.7 | 62.2 | 58.1 | 51.2 |
| | Alkaline peak % | 15.1 | 13.8 | 11.6 | 15.0 | 13.8 | 11.5 | 15.0 | 13.8 | 11.3 | 14.9 | 13.8 | 11.3 |

TABLE 9
Results of a surfactant screening experiment based on vibration

| | | Formulation | | | | | | | | | | | |
|---|---|---|---|---|---|---|---|---|---|---|---|---|---|
| | | F10 | | | F11 | | | F12 | | | F13 | | |
| Vibration day | | 0 | 5 | 7 | 0 | 5 | 7 | 0 | 5 | 7 | 0 | 5 | 7 |
| Appearance | | CC | CC | CC | CC | CC | CC | CC | CC | CC | CC | CC | CC |
| Concentration (mg/mL) | | 30.2 | 30.0 | 30.2 | 30.3 | 30.0 | 30.1 | 30.2 | 30.1 | 30.4 | 30.1 | 30.4 | 30.2 |
| pH | | 5.4 | NA | NA | 5.4 | NA | NA | 5.4 | NA | NA | 5.5 | NA | NA |
| DLS | Particle size (nm) | 4.4 | 4.3 | 4.3 | 4.4 | 4.2 | 4.3 | 4.3 | 4.3 | 4.3 | 4.3 | 4.2 | 4.3 |
| | Polydispersity index % | 5.6 | 4.9 | 6.0 | 5.8 | 6.1 | 4.8 | 5.7 | 5.5 | 4.8 | 4.7 | 5.3 | 4.8 |
| SEC | HMW % | 0.4 | 0.4 | 0.5 | 0.4 | 0.4 | 0.5 | 0.4 | 0.5 | 0.5 | 0.4 | 0.5 | 0.5 |
| | MP % | 99.5 | 99.5 | 99.4 | 99.5 | 99.4 | 99.4 | 99.5 | 99.4 | 99.4 | 99.5 | 99.4 | 99.4 |
| | LMW % | 0.1 | 0.1 | 0.1 | 0.1 | 0.1 | 0.1 | 0.1 | 0.2 | 0.1 | 0.1 | 0.1 | 0.2 |
| NR | HMW % | 0.2 | 0.4 | 0.3 | 0.3 | 0.3 | 0.2 | 0.2 | 0.3 | 0.2 | 0.2 | 0.3 | 0.2 |
| CE-SDS | MP % | 97.1 | 96.8 | 96.9 | 97.0 | 96.9 | 97.0 | 96.8 | 97.0 | 97.1 | 97.0 | 96.6 | 97.0 |
| | LMW % | 2.6 | 2.8 | 2.8 | 2.7 | 2.8 | 2.8 | 3.0 | 2.7 | 2.7 | 2.7 | 3.1 | 2.7 |
| CEX | Acidic peak % | 22.8 | 23.5 | 22.9 | 22.8 | 23.5 | 22.9 | 22.9 | 23.5 | 23.1 | 23.0 | 23.7 | 23.1 |
| | MP % | 62.1 | 62.4 | 62.0 | 62.1 | 62.4 | 62.0 | 62.1 | 62.5 | 62.0 | 62.2 | 62.4 | 62.1 |
| | Alkaline peak % | 15.1 | 14.1 | 15.1 | 15.0 | 14.1 | 15.0 | 15.0 | 14.0 | 14.9 | 14.9 | 13.9 | 14.9 |

### Example 3: Surfactant Screening Experiment-2

### 1. Experimental Design

A surfactant screening experiment was carried out on the anti-CLDN18.2 antibody protein. The anti-CLDN18.2 antibody protein was filtered with a 0.22 μm PES membrane, a sucrose solution and a poloxamer 188 solution were added, and then the anti-CLDN18.2 antibody protein was diluted with a 20 mM histidine buffer with a pH value of 5.5 and prepared into the following formulations. Histidine and sucrose were used as a buffer and a stabilizer, respectively. The final concentration of the anti-CLDN18.2 antibody protein was 30.0 mg/mL. Finally, samples were filtered with a 0.22 μm PES membrane, followed by stability studies listed in Table 10. The samples were tested after incubation.

TABLE 10
Design of a surfactant screening experiment

| | | | Surfactant | |
|---|---|---|---|---|
| | Fixed component of | Surfactant | concentration | Stability conditions and |
| Formula | formula | type | (W/V) | test items |
| F14 | 20 mM histidine and | Poloxamer | 0.05% | An X test, a Y test and a |
| F15 | 9% (W/V) of sucrose, | 188 | 0.10% | Z test described in the |
| F16 | pH 5.5 | | 0.20% | note column were carried |
| | | | | out on the 0 day, and |
| | | | | after incubation was |
| | | | | performed at 40° C. for 3, |
| | | | | 7 or 14 days, the X test |
| | | | | described in the note |
| | | | | column was carried out |
| | | | | on the 3^{rd} day, the 7^{th} day |
| | | | | and the 14^{th} day |
| | | | | separately (T0^{X Y Z} |
| | | | | 40° C. (3 D, 7 D, 14 D)^{X}). |
| | | | | Stirring was performed |
| | | | | (100 rpm: 1 D, 3 D, 5 D)^{X Z}, |
| | | | | and mixing was |
| | | | | performed (300 rpm: |
| | | | | 24 H, 48 H)^{XYZ}. |

Note:
^{X}= Conc., SEC, CEX, DLS, NR-CE-SDS, ^{Y}= MFI, and ^{Z}= visual inspection.

### 2. Analysis of Results

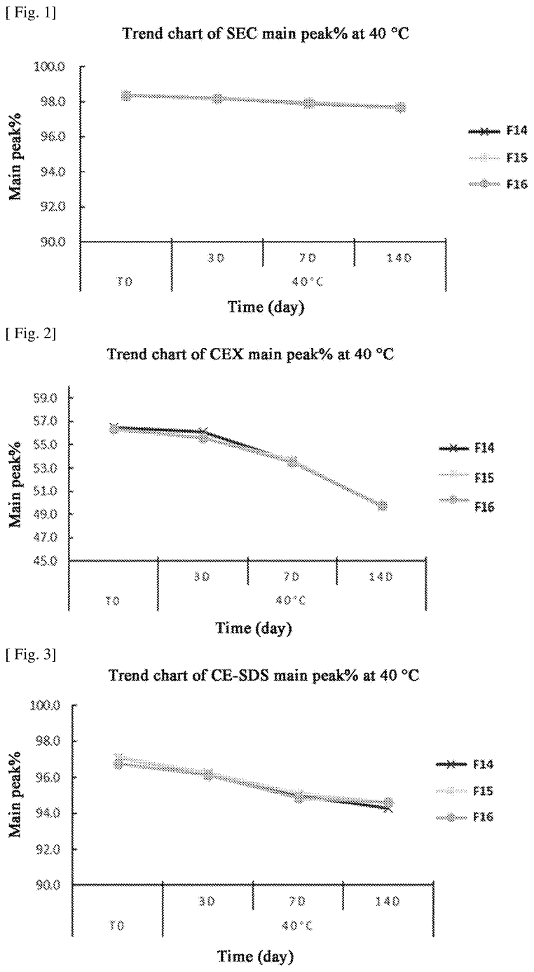

According to the SEC, CE-SDS and CEX data, no obvious differences in the Formulations F14, F15 and F16 were observed (FIGS. 1-3). Thus, the poloxamer 188 surfactant having a concentration of 0.05% (W/V)-0.20% (W/V) also meets the formulation requirements.

### Example 4: Excipient Screening Experiment

### 1. Experimental Design

An excipient screening experiment was carried out on the anti-CLDN18.2 antibody protein. The anti-CLDN18.2 antibody protein was filtered with a 0.22 um PVDF membrane, an excipient solution and a polysorbate 80 solution were added, then the anti-CLDN18.2 antibody protein was prepared into the following formulations, and the formulations were diluted with a 20 mM acetic acid-sodium acetate buffer with a pH value of 5.5 and water. The final concentration of the anti-CLDN18.2 antibody protein was 30.0 mg/mL. Finally, samples were filtered with a 0.22 μm PVDF membrane and respectively loaded into a 2 mL vial packaging container system by a pipette, followed by stability studies listed in Table 11.

TABLE 11
Design of an excipient screening experiment

| | Protein | | | Stability conditions |
|---|---|---|---|---|
| Formula | concentration | Buffer system | Excipient | and test items |
| F17 | 30.0 mg/mL | 20 mM acetate | 150 mM NaCl | An X test, a Y test |
| F18 | | and 0.05% (w/v) | 9% (w/v) of sucrose | and a Z test |
| F19 | | of polysorbate 80, | 9% (w/v) of trehalose | described in the note |
| F20 | | pH 5.5 | 150 mM arginine | column were carried |
| | | | hydrochloride | out on the 0 day, and |
| | | | | after incubation was |
| | | | | performed at 40° C. |
| | | | | for 7 or 14 days, the |
| | | | | X test and the Z test |
| | | | | described in the note |
| | | | | column were carried |
| | | | | out on the 7^{th} day |
| | | | | and the 14^{th} day |
| | | | | respectively (T0^{X Y Z} |
| | | | | 40° C. (7 D, 14 D)^{X}). |
| | | | | After freezing and |
| | | | | thawing were |
| | | | | performed for 5 |
| | | | | cycles, the X test and |
| | | | | the Z test described |
| | | | | in the note column |
| | | | | were carried out (FT |
| | | | | (5x)^{X Z}). |

Note:
1. ^{X}= Conc., SEC, CEX, DLS, NR-CE-SDS, ^{Y}= pH, and ^{Z}= MFI.
2. Considering that the KD values of the formulations F17 and F20 were negative, and that these formulations were slightly opalescent in appearance and had a large radius of single antibody molecules at T0, it is believed that the stability of these formulations was significantly lower than that of other formulations. Therefore, other tests were not carried out on the formulations F17 and F20.

### 2. Analysis of Results

### (1) Freezing and Thawing Test

Results were shown in Table 12. At T0, the Formulations F 17 and F20 were slightly opalescent and colorless liquids in appearance, and the Formulations F 18 and F 19 were clear and colorless liquids. After freezing and thawing were performed for 5 cycles, no obvious changes in the protein concentration and appearance of the Formulations F18 and F19 were observed. At T0, the KD values of the Formulations F17 and F20 were negative, indicating that the action force between single antibody molecules was an attractive force. The KD values of the Formulations F18 and F19 were positive, indicating that the action force between single antibody molecules was a repulsive force. At T0, the radius of single antibody molecules in the Formulations F17 and F20 was significantly larger than that of other formulations. After freezing and thawing was performed for 5 cycles, the radius of single antibody molecules in the Formulations F 18 and F 19 was basically not changed, and unimodal distribution was realized. No significant differences and changes in SEC, NR CE-SDS and CEX main peaks were observed. No significant differences and changes in subvisible particles are observed. Thus, the Formulations F 18 and F 19 meet the experimental requirements.

TABLE 12
Results of an excipient screening experiment based on freezing and thawing

| | | Formula | | | | | | | |
|---|---|---|---|---|---|---|---|---|---|
| | | F17 | | F18 | | F19 | | F20 | |
| Freezing and thawing times | | 0 | 5 | 0 | 5 | 0 | 5 | 0 | 5 |
| Appearance | | SOC | NT | CC | CC | CC | CC | SOC | NT |
| Concentration (mg/mL) | | 29.7 | NT | 29.8 | 29.8 | 29.1 | 29.2 | 30.2 | NT |
| pH | | 5.5 | NT | 5.5 | NT | 5.5 | NT | 5.4 | NT |
| KD (mL/g) | | −6.13 | NT | 9.22 | NT | 10.4 | NT | −5.07 | NT |
| DLS | Particle size (nm) | 6.7 | NT | 4.3 | 4.4 | 4.4 | 4.3 | 6.3 | NT |
| | Polydispersity index % | 3.5 | NT | 6.1 | 6.3 | 6.0 | 7.0 | 3.9 | NT |
| SEC | HMW % | NT | NT | 0.4 | 0.4 | 0.4 | 0.4 | NT | NT |
| | MP % | NT | NT | 99.5 | 99.5 | 99.5 | 99.5 | NT | NT |
| | LMW % | NT | NT | 0.1 | 0.1 | 0.1 | 0.1 | NT | NT |
| NR | HMW % | NT | NT | 0.3 | 0.2 | 0.2 | 0.2 | NT | NT |
| CE-SDS | MP % | NT | NT | 97.0 | 96.9 | 97.1 | 96.9 | NT | NT |
| | LMW % | NT | NT | 2.7 | 2.9 | 2.7 | 2.9 | NT | NT |
| CEX | Acidic peak % | NT | NT | 22.7 | 22.8 | 22.8 | 22.8 | NT | NT |
| | MP % | NT | NT | 62.1 | 62.0 | 62.1 | 62.0 | NT | NT |
| | Alkaline peak % | NT | NT | 15.2 | 15.2 | 15.1 | 15.2 | NT | NT |

### (2) High Temperature Test

Results are as shown in Table 13. After incubation was performed at 40° C. for 7 and 14 days, no significant changes in the protein concentration, appearance and protein particle size of the Formulations F18 and F19 were observed. A small amount of SEC, NR-CE-SDS and CEX main peaks were degraded. However, the acceptable standards were satisfied, and the formulations have no significant differences.

TABLE 13
Results of an excipient screening experiment at 40° C.

| | | Formulation | | | | | |
|---|---|---|---|---|---|---|---|
| | | F18 | | | F19 | | |
| Duration days at 40° C. | | 0 | 7 | 14 | 0 | 7 | 14 |
| Appearance | | CC | CC | CC | CC | CC | CC |
| Concentration (mg/mL) | | 29.8 | 29.8 | 30.0 | 29.1 | 29.2 | 29.4 |
| DLS | Particle | 4.3 | 4.4 | 4.4 | 4.4 | 4.4 | 4.3 |
| | size (nm) | | | | | | |
| | Polydispersity | 6.1 | 4.9 | 5.3 | 6.0 | 5.1 | 4.0 |
| | index % | | | | | | |
| SEC | HMW % | 0.4 | 0.6 | 0.8 | 0.4 | 0.7 | 0.8 |
| | MP % | 99.5 | 99.1 | 96.4 | 99.5 | 99.0 | 96.4 |
| | LMW % | 0.1 | 0.3 | 2.7 | 0.1 | 0.4 | 2.7 |
| NR-CE- | HMW % | 0.3 | 0.2 | 0.6 | 0.2 | 0.2 | 0.7 |
| SDS | MP % | 97.0 | 96.1 | 94.6 | 97.1 | 95.7 | 94.3 |
| | LMW % | 2.7 | 3.7 | 4.8 | 2.7 | 4.1 | 5.0 |
| CEX | Acidic peak % | 22.7 | 27.6 | 36.3 | 22.8 | 27.6 | 36.2 |
| | MP % | 62.1 | 58.3 | 52.0 | 62.1 | 58.4 | 52.1 |
| | Alkaline peak % | 15.2 | 14.1 | 11.7 | 15.1 | 14.0 | 11.6 |

### (3) Conclusion

According to the DLS and appearance results, the excipients such as sodium chloride and arginine hydrochloride were excluded, because the action force between single antibody molecules was changed from a repulsive force to an attractive force after such excipients were added, leading to the risk of aggregation of molecules in long-term storage, which was not conducive to long-term storage of formulations. According to the 40° C. study results and the freezing and thawing study results, the sucrose and the trehalose had basically the same protection effect on the stability of single antibody molecules and both can meet the needs for development of stable formulations.

### Example 5: Excipient Screening Experiment-2

### 1. Experimental Design

An excipient screening experiment was carried out on the anti-CLDN18.2 antibody protein. The anti-CLDN18.2 antibody protein was filtered with a 0.22 μm PES membrane, an excipient solution and a polysorbate 80 solution were added, then the anti-CLDN18.2 antibody protein was prepared into the following formulations, and the formulations were diluted with a 20 mM histidine buffer with a pH value of 5.5. The final concentration of the anti-CLDN18.2 antibody protein was 30.0 mg/mL. Finally, samples were filtered with a 0.22 μm PES membrane, followed by stability studies listed in Table 14.

TABLE 14
Excipient screening experiment-2

| | | | Stability conditions |
|---|---|---|---|
| Formulation | Buffer and surfactant | Excipient | and test items |
| F21 | 20 mM histidine and | 5% (W/V) of sorbitol | T0^{XYZ} |
| F22 | 0.05% (W/V) of | 9% (W/V) of sucrose | 40° C. (3 D, 7 D, 14 D)^{X} |
| | polysorbate 80, pH 5.5 | | FT (1x, 3x, 5x)^{XY} |
| | | | mixing (300 rpm: |
| | | | 24 H, 48 H)^{XYZ}. |

Note:
^{X}= Conc., SEC, CEX, DLS, NR-CE-SDS; ^{Y}= MFI; ^{Z}= visual inspection.

### 2. Analysis of Results

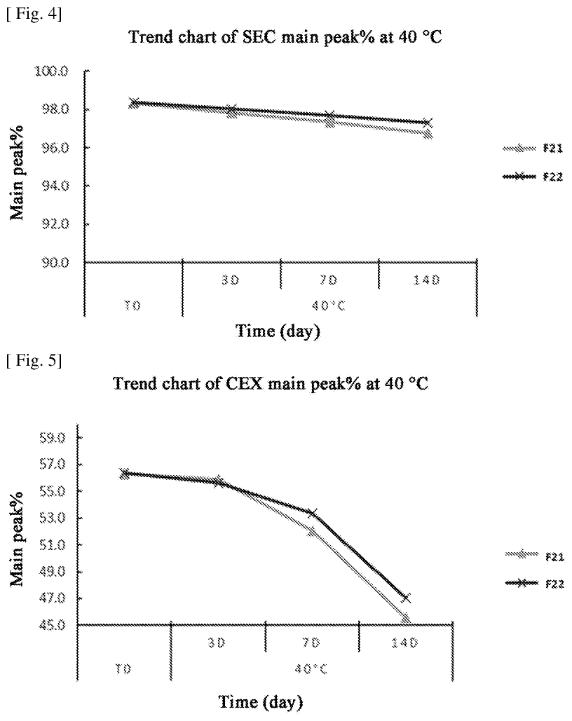

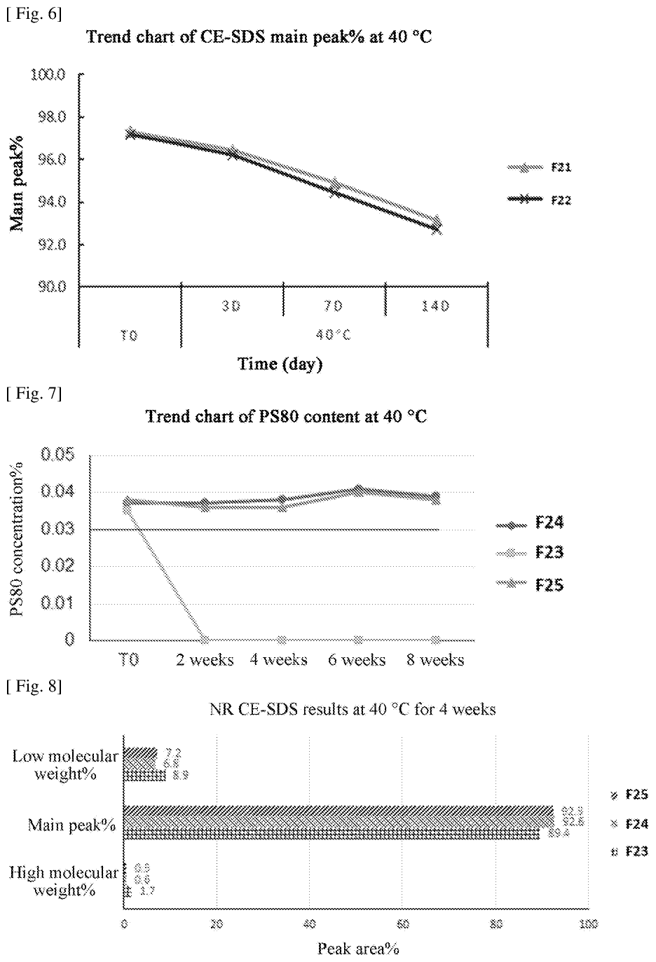

According to a stirring experiment and SEC, CEX and CE-SDS results, both the Formulations F21 and F22 meet the requirements (as shown in FIGS. 4-6 and Table 15).

TABLE 15
Results of an excipient screening experiment-2 based on stirring

| | Appearance | Concentration (mg/mL) | | | pH | |
|---|---|---|---|---|---|---|
| Formulation | T0 | T0 | 24 H | 48 H | T0 | 48 H |
| F21 | Clear and | 30.8 | 31.3 | 31.9 | 5.4 | 5.4 |
| | colorless | | | | | |
| F22 | Clear and | 30.8 | 31.0 | 31.7 | 5.4 | 5.3 |
| | colorless | | | | | |

Therefore, both the sucrose and the sorbitol meet the requirements.

### Example 6: Formulation Verification Experiment

### 1. Experimental Design

Based on the above experiments, 2 formulations meeting the requirements were selected to verify the optimal formulation.

A surfactant solution and an excipient solution were added to a buffer system comprising the anti-CLDN18.2 antibody protein, and the anti-CLDN18.2 antibody protein concentration was adjusted to 30.0 mg/mL to prepare the following formulations. Samples were filtered with a 0.22 μm PVDF membrane and separately loaded into a 2 mL vial packaging container system and a 5 mL polycarbonate flask by a pipette, followed by stability studies.

TABLE 16
Design of a formulation verification study

| Formulation | Description | Stability conditions and test items |
|---|---|---|
| F23 | 30.0 mg/mL of DS, 20 mM acetate, | T0^{XZ} |
| | 9% (w/v) of sucrose and 0.05% | 25° C. (2 W, 4 W, 6 W)^{X} |
| | (w/v) of polysorbate 80, pH 5.3 | 5° C. (2 W, 4 W, 6 W, 3 M, 6 M)^{X} |
| F22 | 30.0 mg/mL of DS, 20 mM | ~20° C. in a PC vial (1 M, 3 M, 6 |
| | histidine, 9% (w/v) of sucrose and | M)^{X} |
| | 0.05% (w/v) of polysorbate 80, pH | |
| | 5.5 | |

Note:
^{X}= Conc., SEC, CEX, NR CE-SDS, visual inspection; ^{Z}= potency.

### 2. Analysis of Results

In an acceleration test (as shown in Table 17), the protein purity (measured by both SEC and NR CE-SDS) was slightly decreased by degradation in an acetic acid buffer system (Formulation F23) and a histidine buffer system (Formulation F22), and a protein was degraded to a lower degree in the acetic acid buffer system (Formulation F23). The CEX purity was not significantly changed in the acetic acid buffer system, but was significantly changed in the histidine buffer system. In a long-term test (as shown in Table 18) and a low temperature test (as shown in Table 19), the acetic acid buffer system (Formulation F23) and the histidine buffer system (Formulation F22) were not significantly changed in appearance, pH, concentration, DLS and purity (including SEC, NR CE-SDS and CEX). Considering that the degradation trend of a protein under long-term storage conditions can be better simulated by the conditions of the acceleration test, the acetic acid buffer system (Formulation F23) which was more stable in the acceleration test was selected as the final formulation which included 30.0 mg/mL of an anti-CLDN18.2 antibody protein, 20 mM acetic acid/sodium acetate, 9% (w/v) of sucrose and 0.05% (w/v) of polysorbate 80 and has a pH value of 5.3.

TABLE 17
Acceleration test at 25° C.

| | | Formulation | | | | | | | |
|---|---|---|---|---|---|---|---|---|---|
| | | F23 | | | | F22 | | | |
| Duration weeks at 25° C. | | 0 | 2 | 4 | 6 | 0 | 2 | 4 | 6 |
| Appearance | | CC | CC | CC | CC | CC | CC | CC | CC |
| Concentration (mg/mL) | | 30.2 | 30.5 | 30.4 | 30.4 | 30.3 | 30.4 | 30.4 | 30.6 |
| SEC | HMW % | 0.4 | 0.5 | 0.5 | 0.6 | 0.4 | 0.6 | 0.6 | 0.9 |
| | MP % | 99.4 | 99.3 | 99.3 | 99.1 | 99.4 | 99.3 | 99.1 | 98.7 |
| | LMW % | 0.2 | 0.1 | 0.2 | 0.3 | 0.2 | 0.2 | 0.3 | 0.3 |
| NR-CE-SDS | HMW % | 0.1 | 0.2 | 0.4 | 0.3 | 0.1 | 0.3 | 0.4 | 0.4 |
| | MP % | 97.0 | 96.6 | 96.4 | 96.2 | 97.1 | 96.4 | 95.8 | 95.6 |
| | LMW % | 3.0 | 3.2 | 3.3 | 3.5 | 2.9 | 3.4 | 3.8 | 3.9 |
| CEX | Acidic peak % | 17.0 | 16.5 | 18.0 | 18.7 | 17.3 | 17.9 | 22.4 | 25.6 |
| | MP % | 59.7 | 59.6 | 59.6 | 58.0 | 59.6 | 52.9 | 44.2 | 39.9 |
| | Alkaline peak % | 23.3 | 23.9 | 22.4 | 23.2 | 23.1 | 29.2 | 33.4 | 34.5 |

TABLE 18
Results of a formulation verification experiment at 5° C.

| | | Formulation | | | | | | | | | | | |
|---|---|---|---|---|---|---|---|---|---|---|---|---|---|
| | | F23 | | | | | | F22 | | | | | |
| Duration time at 5° C. | | T0 | 2 W | 4 W | 6 W | 3 M | 6 M | T0 | 2 W | 4 W | 6 W | 3 M | 6 M |
| Appearance | | CC | CC | CC | CC | CC | CC | CC | CC | CC | CC | CC | CC |
| Concentration (mg/mL) | | 30.2 | 30.6 | 30.5 | 30.3 | 30.5 | 30.1 | 30.3 | 30.4 | 30.2 | 30.4 | 30.4 | 30.2 |
| DLS | Radius (nm) | 4.0 | 4.1 | NT | 4.0 | NT | 4.2 | 4.6 | 4.0 | NT | 4.7 | NT | 4.8 |
| | PDI % | 4.3 | 5.1 | NT | 4.5 | NT | 9.4 | 4.6 | 5.2 | NT | 4.4 | NT | 9.4 |
| SEC | HMW % | 0.4 | 0.4 | 0.4 | 0.5 | 0.7 | 0.6 | 0.4 | 0.4 | 0.3 | 0.5 | 0.7 | 0.7 |
| | MP % | 99.4 | 99.5 | 99.5 | 99.4 | 99.2 | 99.3 | 99.4 | 99.5 | 99.5 | 99.4 | 99.1 | 99.0 |
| | LMW % | 0.2 | 0.1 | 0.1 | 0.2 | 0.1 | 0.2 | 0.2 | 0.1 | 0.1 | 0.2 | 0.2 | 0.3 |
| NR-CE- | HMW % | 0.1 | 0.1 | 0.2 | 0.1 | 0.1 | 0.2 | 0.1 | 0.2 | 0.2 | 0.1 | 0.6 | 0.2 |
| SDS | MP % | 97.0 | 96.8 | 96.9 | 97.0 | 96.8 | 96.8 | 97.1 | 97.0 | 96.7 | 97.2 | 96.1 | 96.4 |
| | LMW % | 3.0 | 3.0 | 2.9 | 2.9 | 3.1 | 3.1 | 2.9 | 2.8 | 3.1 | 2.7 | 3.4 | 3.4 |
| CEX | Acidic peak % | 17.0 | 15.6 | 16.3 | 15.6 | 16.8 | 15.9 | 17.3 | 16.1 | 16.6 | 16.1 | 17.9 | 18.6 |
| | MP % | 59.7 | 59.8 | 60.0 | 59.3 | 61.2 | 60.0 | 59.6 | 59.6 | 60.0 | 59.1 | 60.0 | 56.8 |
| | Alkaline peak % | 23.3 | 24.6 | 23.8 | 25.0 | 22.0 | 24.2 | 23.1 | 24.4 | 23.4 | 24.9 | 22.1 | 24.5 |

TABLE 19
Results of a formulation verification experiment at −20° C.

| | | Formulation | | | | | | | |
|---|---|---|---|---|---|---|---|---|---|
| | | F23 | | | | F22 | | | |
| Duration month at −20° C. | | T0 | 1 M | 3 M | 6 M | T0 | 1 M | 3 M | 6 M |
| Concentration (mg/mL) | | 30.2 | 30.2 | 30.8 | NT | 30.3 | 30.3 | 30.5 | NT |
| SEC | HMW % | 0.4 | 0.3 | 0.5 | 0.5 | 0.4 | 0.3 | 0.5 | 0.4 |
| | MP % | 99.4 | 99.6 | 99.4 | 99.4 | 99.4 | 99.6 | 99.4 | 99.5 |
| | LMW % | 0.2 | 0.1 | 0.1 | 0.1 | 0.2 | 0.1 | 0.1 | 0.1 |
| NR-CE-SDS | HMW % | 0.1 | 0.1 | 0.1 | 0.1 | 0.1 | 0.1 | 0.5 | 0.2 |
| | MP % | 97.0 | 96.7 | 97.1 | 97.0 | 97.1 | 96.6 | 96.3 | 96.9 |
| | LMW % | 3.0 | 3.2 | 2.9 | 2.9 | 2.9 | 3.3 | 3.2 | 2.9 |
| CEX | Acidic peak % | 17.0 | 16.7 | 16.9 | 15.5 | 17.3 | 16.9 | 17.3 | 15.9 |
| | MP % | 59.7 | 60.6 | 61.3 | 60.0 | 59.6 | 60.6 | 61.2 | 59.8 |
| | Alkaline peak % | 23.3 | 22.7 | 21.8 | 24.5 | 23.1 | 22.5 | 21.5 | 24.3 |

### Example 7: Investigation of a Protection Effect of Disodium Edetate (EDTA-Na₂)

### 1. Experimental Design

The following samples as shown in the table below were prepared, and a protection effect of disodium edetate on preventing the degradation of polysorbate 80 (PS80) and maintaining the protein quality was investigated on the Formulations F23 and F24 (for F24, EDTA was added into a stock solution, shaking was conducted for uniform mixing with the stock solution, the EDTA was removed by a dialysis method, and a final formulation was formulated) and the Formulation F25. The Formulation F24 was prepared by the following method: EDTA was added into an antibody stock solution, the antibody stock solution was subjected to solvent exchange with a formulation solvent comprising a buffer and a stabilizer to achieve the effects of removing the EDTA and realizing solvent exchange by filtration simultaneously, and finally a surfactant was added to obtain a final formulation.

TABLE 20
Experimental design of a disodium edetate protection study

| | | Stability |
|---|---|---|
| | | conditions and |
| Formulation | Description | test items |
| F23 | 30.0 mg/mL of DS, 20 mM acetate, 9% (w/v) of | T0, |
| | sucrose and 0.05% (w/v) of polysorbate 80, pH 5.3 | 40° C.: |
| | (control group) | 2 W/4 W/6 W/8 W |
| F24 | 30.0 mg/mL of DS, 20 mM acetate, 9% (w/v) of | Visual, Conc., |
| | sucrose and 0.05% (w/v) of polysorbate 80, pH 5.3 | OD405, DLS, |
| | (EDTA was added into a stock solution and removed | SEC, PS80 content, |
| | by dialysis) | NR CE (4 W) |
| F25 | 30.0 mg/mL of DS, 20 mM acetate, 9% (w/v) of | |
| | sucrose, 0.05% (w/v) of polysorbate 80 and 50 μM of | |
| | EDTA, pH 5.3 (the formulation includes EDTA) | |

### 2. Analysis of Results

Under the forced experimental conditions at 40° C., it can be seen from FIG. 7 that the degradation of PS80 could be effectively prevented by either adding EDTA during the downstream purification (Formulation F24) or adding the EDTA to a formulation (Formulation F25). It can be seen from FIG. 8 that the degradation of main peaks of the protein was reduced by the formulations F24 and F25.

1. A pharmaceutical formulation, comprising an anti-CLDN18.2 antibody and a buffer, wherein the buffer is an acetic acid buffer or a histidine buffer, and the pH value of the pharmaceutical formulation is 4.5-6.0.

2. The pharmaceutical formulation according to claim 1, wherein the concentration of the buffer in the pharmaceutical formulation is 5 mM-50 mM, or 10 mM-30 mM.

3. The pharmaceutical formulation according to claim 1, further comprising a stabilizer, a surfactant, or a chelating agent.

4. The pharmaceutical formulation according to claim 3, wherein:
(i) the concentration of the stabilizer in the pharmaceutical formulation is 1% (w/v)-20% (w/v) or 1% (w/v)-10% (w/v);
(ii) the concentration of the surfactant in the pharmaceutical formulation is 0.005% (w/v)-0.4% (w/v) or 0.01% (w/v)-0.2% (w/v); or
(iii) the concentration of the chelating agent in the pharmaceutical formulation is 30 μM-350 μM or 40 μM-60 μM.

5. The pharmaceutical formulation according to claim 3, wherein:
(i) the stabilizer is selected from the group consisting of: sucrose, trehalose, and sorbitol;
(ii) the surfactant is selected from the group consisting of: polysorbate 80 and poloxamer 188; or
(iii) the chelating agent is selected from the group consisting of: EDTA, DTPA, IDHA, EDDHA and HBED.

6. The pharmaceutical formulation according to claim 5, wherein
(a) the stabilizer is sucrose or trehalose, and the concentration of the sucrose or the trehalose in the pharmaceutical formulation is 5% (w/v)-10% (w/v); or
(b) the stabilizer is sorbitol, and the concentration of the sorbitol in the pharmaceutical formulation is 2% (w/v)-8% (w/v).

7-9. (canceled)

10. The pharmaceutical formulation according to claim 5, wherein
(a) the surfactant is polysorbate 80, and the concentration of the polysorbate 80 in the pharmaceutical formulation is 0.01% (w/v)-0.1% (w/v); or
(b) the surfactant is poloxamer 188, and the concentration of the poloxamer 188 in the pharmaceutical formulation is 0.05% (w/v)-0.2% (w/v).

11-13. (canceled)

14. The pharmaceutical formulation according to claim 1, wherein the concentration of the anti-CLDN18.2 antibody in the pharmaceutical formulation is 1 mg/ml-200 mg/ml or 20 mg/ml-40 mg/ml.

15. (canceled)

16. The pharmaceutical formulation according to claim 1, wherein the anti-CLDN18.2 antibody comprises:
(a) a heavy chain CDR1 (HCDR1) as set forth in SEQ ID NO: 1, an HCDR2 as set forth in SEQ ID NO: 2 and an HCDR3 as set forth in SEQ ID NO: 3, and a light chain CDR1 (LCDR1) as set forth in SEQ ID NO: 4, an LCDR2 as set forth in SEQ ID NO: 5 and an LCDR3 as set forth in SEQ ID NO: 6;
(b) a CDR1, a CDR2 and a CDR3 of the heavy chain variable region as set forth in SEQ ID NO: 7, and a CDR1, a CDR2 and a CDR3 of the light chain variable region as set forth in SEQ ID NO: 8;
(c) a heavy chain variable region as set forth in SEQ ID NO: 7 and a light chain variable region as set forth in SEQ ID NO: 8; or
(d) a heavy chain as set forth in SEQ ID NO: 9 and a light chain as set forth in SEQ ID NO: 10.

17-19. (canceled)

20. The pharmaceutical formulation according to claim 1, comprising the anti-CLDN18.2 antibody, the buffer, a stabilizer and a surfactant, wherein:
(a) the buffer is an acetic acid buffer, the stabilizer is sucrose or trehalose, the surfactant is polysorbate 80, and the pH value of the pharmaceutical formulation is about 4.5-6.0; or
(b) the buffer is a histidine buffer, the stabilizer is sucrose or sorbitol, and the surfactant is polysorbate 80 or poloxamer 188.

21. The pharmaceutical formulation according to claim 20, wherein:
(a) the concentration of the anti-CLDN18.2 antibody in the pharmaceutical formulation is 20 mg/ml-40 mg/ml, the concentration of the acetic acid buffer in the pharmaceutical formulation is 10 mM-30 mM, the concentration of the sucrose or the trehalose in the pharmaceutical formulation is 5% (w/v)-10% (w/v), and/or the concentration of the polysorbate 80 in the pharmaceutical formulation is 0.01% (w/v)-0.2% (w/v); or
(b) the concentration of the anti-CLDN18.2 antibody in the pharmaceutical formulation is about 30 mg/ml, the concentration of the acetic acid buffer in the pharmaceutical formulation is about 20 mM, the concentration of the sucrose or the trehalose in the pharmaceutical formulation is about 9% (w/v), the concentration of the polysorbate 80 in the pharmaceutical formulation is 0.010% (w/v)-0.1% (w/v), and the pH value is about 5.0-5.5.

22. (canceled)

23. The pharmaceutical formulation according to claim 21, wherein the stabilizer is sucrose, the concentration of the polysorbate 80 in the pharmaceutical formulation is about 0.05% (w/v), and the pH value is about 5.3.

24. The pharmaceutical formulation according to claim 20, further comprising EDTA, wherein:
(a) the concentration of the EDTA in the pharmaceutical formulation is 40 μM-60 μM; or
(b) the concentration of the EDTA in the pharmaceutical formulation is about 50 μM.

25-26. (canceled)

27. The pharmaceutical formulation according to claim 20, wherein:
(a) the stabilizer is sucrose, and the concentration of the sucrose in the pharmaceutical formulation is 6% (w/v)-12% (w/v);
(b) the stabilizer is sorbitol, and the concentration of the sorbitol in the pharmaceutical formulation is 2% (w/v)-8% (w/v);
(c) the surfactant is polysorbate 80, and the concentration of the polysorbate 80 in the pharmaceutical formulation is 0.01% (w/v)-0.1% (w/v); or
(d) the surfactant is poloxamer 188, and the concentration of the poloxamer 188 in the pharmaceutical formulation is 0.05% (w/v)-0.2% (w/v).

28-30. (canceled)

31. A method for preparing a pharmaceutical formulation, comprising:
1) providing a formulation solvent and an anti-CLDN18.2 antibody stock solution, wherein the formulation solvent comprises a buffer, a stabilizer and optionally a surfactant; and
2) subjecting the anti-CLDN18.2 antibody stock solution to solvent exchange with the formulation solvent to obtain the pharmaceutical formulation according to claim 1.

32. The method according to claim 31, wherein the anti-CLDN18.2 antibody stock solution comprises a chelating agent (e.g., EDTA), the formulation solvent does not comprise a chelating agent, and the pharmaceutical formulation comprises substantially no chelating agent after filtration.

33. The method according to claim 32, wherein the formulation solvent comprises a buffer and a stabilizer and does not comprise a surfactant.

34. The method according to claim 31, wherein the formulation solvent comprises a buffer, a stabilizer and a chelating agent (e.g., EDTA).

35. The method according to claim 34, wherein the surfactant is added after the anti-CLDN18.2 antibody stock solution has been subjected to solvent exchange with the formulation solvent to obtain the pharmaceutical formulation.

36. The pharmaceutical formulation according to claim 1, wherein the acetic acid buffer is an acetic acid-sodium acetate buffer; and the histidine buffer is a histidine-histidine hydrochloride buffer.

37. A method of preventing and/or treating CLDN18.2 related diseases in a subject, comprising administrating the pharmaceutical formulation according to claim 1 to the subject.

38. The method according to claim 37, wherein the CLDN 18.2 related diseases are selected from the group consisting of: gastric cancer, adenocarcinoma of the gastroesophageal junction, lung cancer, bronchogenic carcinoma, bone cancer, hilarcholangiocarcinoma, pancreatic cancer, breast cancer, liver cancer, ovarian cancer, testicular cancer, kidney cancer, bladder cancer, head and neck cancer, spinal cancer, brain cancer, cervical cancer, uterine cancer, endometrial cancer, colon cancer, colorectal cancer, rectal cancer, anal cancer, esophageal carcinoma, gastrointestinal cancer, skin cancer, prostate cancer, pituitary carcinoma, vaginal cancer, thyroid cancer, glioblastoma, astrocytoma, melanoma, myelodysplastic syndrome, sarcoma, teratoma or adenocarcinoma.
